(19) United States
(12) Patent Application Publication
(10) Pub. No.: US 2003/0105046 A1
(43) Pub. Date: Jun. 5, 2003

(54) Interleukin converting enzyme (ICE) and central nervous system damage

(75) Inventors: Junying Yuan, Newton, MA (US); Robert M. Friedlander, Cambridge, MA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: The General Hospital Corporation

(21) Appl. No.: 10/123,529

(22) Filed: Apr. 17, 2002

Related U.S. Application Data
(63) Continuation of application No. 09/099,463, filed on Jun. 18, 1998, now abandoned.
(60) Provisional application No. 60/050,242, filed on Jun. 19, 1997.

(51) Int. Cl. A61K 48/00

(52) U.S. Cl. 514/44

(57) ABSTRACT

The invention relates to methods of treating central nervous system damage. This includes methods of treating ALS using a mutant ICE gene and methods of treating head trauma injuries by ICE inhibition. The invention also relates to transgenic non-human animals comprising a mutant ICE gene and a mutant SOD gene. These transgenic animals exhibit attenuated symptoms of ALS. This invention also relates to methods of using the transgenic animals to screen for compounds to treat ALS.

### STATEMENT AS TO RIGHTS TO INVENTIONS MADE UNDER FEDERALLY-SPONSORED RESEARCH AND DEVELOPMENT

Part of the work performed during the development of this invention was supported by U.S. Government funds. The U.S. Government may have certain rights in this invention.

This application claims the benefit of the filing date of U.S. Provisional Application No. 60/050,242 filed Jun. 19, 1997 and said provisional application is incorporated herein by reference.

### BACKGROUND OF THE INVENTION

1. Field of the Invention

The invention is generally in the field of molecular biology as related to the control of programmed cell death and treatment of disease. The invention further relates to treatment of amyotrophic lateral sclerosis and head trauma injury.

2. Related Art

Amyotrophic Lateral Sclerosis (ALS)

Amyotrophic lateral sclerosis (ALS), is a progressive age-dependent disease involving degeneration of motor neurons in the brain, brain stem and spinal cord. Both familial and sporadic forms of this disease exist. In the most frequent form of ALS, patients evidence both lower motor neuron impairment (including muscular atrophy and weakness) as well as upper motor neuron damage. The principal pathologic changes are loss of motor neurons and their axons, with very little gliotic reaction. Related variants of ALS include progressive bulbar palsy, progressive muscular atrophy and primary lateral sclerosis. (Robbins et al., Basic Pathology, W. B. Saunder Co. (1987))

ALS is characterized by neuronal cell death. Little is known about the triggering mechanism responsible for executing this cell death in ALS. Although ALS has been included in a list of diseases associated with increased apoptosis (Thompson, C. B., Science 267:1456-1462 (1995), there has been no direct evidence in the art to indicate that such is actually the case.

Mutations in the Cu/Zn superoxide dismutase (SOD-1) gene have been shown to be responsible for some of the familial forms of ALS (Rosen, D. R., et al., Nature 362:59-62 (1993)). Additionally, evidence described by Rothstein et al. (Proc. Natl. Acad. Sci. USA 91:4155-9 (1994)) demonstrated that down regulation of SOD-1 activity using antisense SOD-1 in vitro promotes apoptosis in a neuronal cell line. Cell death in the neuronal cell line was mediated in part by the activation of the Interleukin-1β converting enzyme (ICE), and by binding of endogenously produced mature IL-1β to its receptor (Troy, C. M., et al., Proc. Natl. Acad. Sci. USA 93:563540 (1996)). Therefore, a better understanding of the role of cell death and what triggers such death in ALS would lead to a more rational treatment and possible cure for the disease.

Traumatic Brain Injury (TBI)

Traumatic injury is the third leading cause of death in the western world, superseded only by cancer and heart disease. Half of traumatic deaths are directly attributed to brain injury (Waxweiler, R. J., et al., J. Neurotrauma 12:509-516 (1995); Department of Health and Human Services, Interagency Head Injury Task Force Report (1989)). Despite the societal impact of traumatic brain injury (TBI), little is known regarding the basic mechanistic pathways by which it mediates cell death. Traditional notion attributes cell death to necrosis as the major mechanism following TBI (Shapira, Y., et al., Crit. Care Med. 16:258-265 (1988); Dietrich, W. D., et al., Acta Neuropathol. 87:250-258 (1994)). This view has been recently challenged with the detection of apoptotic cells in experimental brain injury models (Rink, A., et al., Am. J. Pathol. 147:1575-1583 (1995); Colicos, M. A. & Dash, P. K., Brain Res. 739:120-131 (1996); Yakovlev, A. G. et al., J. Neurosci. 17:7415-7424 (1997); Sinson, G., et al., J. Neurosurg. 86:511-518 (1997)), as well as in humans following head trauma (Thomas, L. B., et al., Exp. Neurol. 133:265-272 (1995)). Understanding the pathways mediating posttraumatic apoptosis might lead to novel approaches to rational pharmacotherapy of TBI.

Programmed Cell Death

Apoptosis, also referred to as programmed cell death or regulated cell death, is a process by which organisms eliminate unwanted cells. Such cell death occurs as a normal aspect of animal development as well as in tissue homeostasis during aging and in disease (Glucksmann, A., Biol. Rev. Cambridge Philos. Soc. 26:59-86 (1950); Ellis et al., Dev. 112:591-603 (1991); Vaux et al., Cell 76:777-779 (1994); Thompson, C. B., Science 267:1456-1462 (1995))). Programmed cell death can also act to regulate cell number, to facilitate morphogenesis, to remove harmful or otherwise abnormal cells and to eliminate cells that have already performed their function. Additionally, programmed cell death is believed to occur in response to various physiological stresses such as hypoxia or ischemia. The morphological characteristics of apoptosis include plasma membrane blebbing, condensation of nucleoplasm and cytoplasm and degradation of chromosomal DNA at inter-nucleosomal intervals. (Wyllie, A. H., in Cell Death in Biology and Pathology, Bowen and Lockshin, eds., Chapman and Hall (1981), pp. 9-34).

Apoptosis is achieved through an endogenous mechanism of cellular suicide (Wyllie, A. H., in Cell Death in Biology and Pathology, Bowen and Lockshin, eds., Chapman and Hall (1981), pp. 9-34) and occurs when a cell activates its internally encoded suicide program as a result of either internal or external signals. The suicide program is executed through the activation of a carefully regulated genetic program (Wylie, A. H., et al., Int. Rev. Cyt. 68: 251 (1980); Ellis, R. E., et al., Ann. Rev. Cell Bio. 7: 663 (1991); Yuan, Y. Curr. Op. Cell. Biol. 7:211-214 (1995)). In many cases, gene expression appears to be required for apoptosis, since cell death can be prevented by inhibitors of RNA or protein synthesis (Cohen et al., J. Immunol. 32:3842 (1984); Stanisic et al., Invest. Urol. 16:19-22 (1978); Martin et al., J. Cell Biol. 106:829-844 (1988).

Acute and chronic disregulation of cell death is believed to lead to a number of major human diseases (Barr et al. Biotech. 12:487-493 (1995); Thompson C. B., Science 267:1456-1462 (1995)). These diseases include but are not limited to malignant and pre-malignant conditions, neurological and neurodegenerative disorders, heart disease, immune system disorders, intestinal disorders, kidney disease, aging, viral infections and AIDS.

Malignant and pre-malignant conditions may include solid tumors, B cell lymphomas, chronic lymphocytic leukemia, prostate hypertrophy, preneoplastic liver foci and resistance to chemotherapy. Neurological disorders may include stroke, Alzheimer's disease, amyotrophic lateral sclerosis, prion-associated disorder and ataxia telangiectasia. Heart disease may include ischemic cardiac damage and chemotherapy-induced myocardial suppression. Immune system disorder may include AIDS, type I diabetes, lupus erythematosus, Sjogren's syndrome and glomerulonephritis. Intestinal disorder may include dysentery, inflammatory bowel disease and radiation- and HIV-induced diarrhea. Kidney disease may include polycystic kidney disease and anemia/erythropoiesis. Specific references to many of these pathophysiological conditions as involving disregulated apoptosis can be found in Barr et al. Id.—Table I.

Interleukin-1-β Converting Enzyme (ICE or Caspase) Family

Mechanistically, apoptotic cell death is mediated by a family of cysteine proteases known as caspases (Alnemri, E. S., et al., Cell 87:171 (1996)). Caspases are mammalian homologues of the C. elegans death gene product CED-3 (Yuan, J. & Horvitz, H. R., Dev. Biol. 138:33-41 (1990); Yuan, J., Shaham, et al., Cell 75:641-652 (1993)) which execute, together with CED-4, apoptotic cell death in the nematode. Interleukin-1β converting enzyme (ICE; caspase-1), the first identified member of the mammalian caspase family, is a cysteine protease responsible for the activation of pro-IL-β (Yuan, J., Shaham, et al., Cell 75:641-652 (1993); Ceretti, D. P., et al., Science 256:97-100 (1992); Miura, M., Cell 75:653-660 (1993)). The involvement of ICE in apoptosis has been demonstrated in a variety of experimental paradigms (Miura, M., Cell 75:653-660 (1993); Gagliardini, V., et al., Science 263:826-828 (1994); Los, M., et al., Nature 375:81-83 (1995); Enari, M., et al., Nature 380:723-726 (1996)). ICE activation, as demonstrated by the detection of mature IL-1βb, has been identified during apoptosis both in vitro as well as in vivo (Hogquist, K. A., et al., Proc. Natl. Acad. Sci. USA 88:8485-8489 (1991); Zychlinsky, A., et al., J. Clin. Invest. 94:1328-1332 (1994); Miura, M., et al., Proc. Natl. Acad. Sci. USA 92:8318-8322 (1995); Hara, H., et al., Proc. Natl. Acad. Sci. USA 94:2007-2012 (1997); Hara, H., et al., J. Cereb. Blood Flow Metab. 17:370-375 (1997)). It has previously been shown that endogenously produced mature IL-1βb, processed following ICE activation, plays an important role in apoptosis (Friedlander, R. M., et al., J. Exp. Med. 184:717-724 (1996); Troy, C. M., et al., Proc. Natl. Acad. Sci. USA 93:5635-5640 (1996)).

ICE is a cytoplasmic cysteine protease responsible for proteolytic processing of pro-interleukin-1β (31 kDa) into its active form (17 kDa) (Thomberry, N. A., Nature 356:768-774 (1992), Cerretti, D. P., et al., Science 256:97-100 (1992)). ICE is synthesized as a precursor of 45 kDa which is proteolytically cleaved during activation to generate two subunits of 22 kDa (p20) and 10 kDa (p10) (Thornberry, N. A., et al, Nature 356:768-774 (1992)).

ICE is a member of a large family of apoptotic gene products. The ICE family (Caspase family) comprises an increasing number of cysteine proteases involved in cytokine maturation and apoptosis (Yuan, J., Curr. Opin. in Cell Biology 7:211-214 (1995)). To date, ten ICE homologs of human origin have been published and the family members are now also referred to as “caspases.” (Alnemri et al., Cell 87:171 (1996)). ICE is referred to as caspase-1 (CASP-1). Frequently, the murine caspases may be found to have the designation mCASP. (Van de Craen et al., FEBS Lett. 401:61-69 (1997))

The mammalian ICE/CED-3 family includes eight members ICE, TX/ICEᵣₑₗII/ICH-2, ICEᵣₑₗIII, ICH-1/NEDD2, CPP32/Yama/Apopain, MCH2, MCH-3/ICE-LAP3/MCH-2 and ICH-3 (Kumar et al., Genes Dev. 8:1613-1626 (1994); Femandes-Alnemri, et al., J. Biol. Chem. 269:30761-30764 (1994); Femandez-Alnemri et al., Cancer Res. 55:2737-2742(1995); Fernandes-Alnemri et al., Cancer Res. 55:6045-6052 (1996); Wang et al., Cell 78:739-750 (1994); Faucheu, et al., EMBO J. 14:1914-1922 (1995); Tewari & Dixit, J. Biol. Chem. 270:3255-3260 (1995); Kamens et al., J. Biol. Chem. 270:15250-15256 (1995); Munday, N. A., et al., J. Biol. Chem. 270:15870-15876 (1995); Duan, H. J., et al., J. Biol. Chem. 271:1621-1625 (1996); Lippke, J. A., et al., J. Biol. Chem. 271:1825-1828 (1996)). All of the above have more recently been designated as caspases. A comparison of ICE and ced-1 is shown in FIGS. 1A-1B.

X-ray crystallography analysis of three dimensional structure of ICE showed that ICE is a dimer of activated ICE p20 and p10 subunits (Wilson, K. P., et al., Nature 370:270-275 (1994); Walker, N. P. C., et al., Cell 78:343-352 (1994)). Activated ICE can cleave the inactive ICE precursor; however, in vitro synthesized ICE precursor cannot cleave itself (Thornberry, N. A., et al., Nature 356:768-774 (1992)), suggesting that ICE may need to be activated by another protease in vivo.

The amino acid sequence of ICE shares 29% identity with C. elegans cell death gene product Ced-3 (Yuan et al., Cell 75:641-752 (1993)) which suggests that ICE may play a role in controlling mammalian apoptosis. In this regard, it has been demonstrated that ICE-mediated endogenously produced mature IL-1β plays an important role in a variety of cell death paradigms (Friedlander, R. M., et al., J. Exp. Med. 184:717-724 (1996)).

Expression of ICE in a number of mammalian cell lines induces apoptosis (Miura et al., Cell 75:653-660 (1993); Wang et al., Cell 87:739-750 (1994)). Microinjection of an expression vector of crmA, a cowpox virus gene encoding a serpin that is a specific inhibitor of ICE, prevents the death of neurons from the dorsal root ganglia and ciliary ganglia, when such death is induced by trophic factor deprivation (Gagliardini et al., Science 263:826-828 (1994); Li et al., Cell 80:401411 (1995); Allsopp et al., Cell 73:295-307, (1993)). Expression of crmA can also suppress apoptosis induced by TNF-α and Fas (Enari et al., Nature 375:78-81 (1995); Los et al., Nature 375:81-83 (1995); Kuide et al., Science 267:2000-2002 (1995); Miura et al., Proc. Natl. Acad. Sci. USA 92:8318-8322 (1995)). These experiments suggest that the members of the ICE family (Caspase family) play important roles in controlling mammalian apoptosis. These results did not indicate, however, which member of the ICE family (Caspase family) is critical for cell death since CrmA may cross-inhibit other members of the ICE family.

The control of apoptosis in mammals is much more complex than that in C. elegans where function of one ced-3 gene controls all programmed cell death (Ellis & Horvitz, Cell 44:817-829 (1986)). In contrast to C. elegans, multiple proteases may be involved in regulation of programmed cell death (apoptosis) in mammals. This hypothesis is supported by many in vitro studies. For instance, peptide inhibitors of ICE such as YVAD-cmk inhibit Fas induced apoptosis but require much higher doses than that for inhibiting ICE (Enari et al., Nature 375:78-81 (1995)), suggesting that inhibition of additional ICE-like protease(s) is required for complete inhibition of Fas induced apoptosis. Similarly, Ac-DEVD-CHO, a peptide inhibitor of CPP32/Yama/Apopain, inhibits poly(ADP-ribose) polymerase (PARP) cleavage at a dose of 1 nM but requires 1 μM to cause 50% inhibition of apoptosis in an cell-free system (Nicholson, D. W., et al., Nature 376:3743 (1995)), suggesting that inhibition of protease(s) other than CPP32/Yama/Apopain is required for complete inhibition of apoptosis in this system. Furthermore, inhibitors that are known not to have effects or have little effects on ICE-like cysteine proteases such as cysteine protease inhibitors trans-epoxysuccininyl-L-leucylamido-(4-guanidino) butane (E64) and leupeptin, calpain inhibitors I and II, and serine protease inhibitors diisopropyl fluorophosphate and phenylmethylsulfonyl fluroride, were found to inhibit apoptosis induced by T cell receptor binding-triggered apoptosis (Sarin et al., J. Exp. Med. 178:1693-1700 (1993)). This suggests that not only cysteine proteases but also serine proteases may play important roles in mammalian cell apoptosis.

Additionally, ICE may also be involved in γ-irradiation induced cell death in concanavalin A (conA)-stimulated splenocytes (Tamura et al., Nature 376:596-599 (1995)). Expression of ICE is induced in splenocytes stimulated by conA and induction of Ice expression enhances the susceptibility of mitogen activated T cells to cell death induced by γ-irradiation and DNA-damaging chemotherapeutic agents such as adriamycin or etoposide induced cell death.

Knowing the genes and substrates involved in the ICE pathway and effects of altering or eliminating expression of apoptotic proteins such as ICE or ICH-3 leads to means for modulating (i.e. increasing or decreasing) cell death thereby altering apoptosis. A better understanding of the apoptosis pathways and specific gene products such as ICE can also lead to development of assays for agents which may affect the apoptotic process, and thereby lead to therapies for disease treatments. Interventions may include, inter alia, agents which affect the activities of the gene products (e.g. agents which block receptors, inhibit or stimulate enzymatic activity), modulation of the gene product using gene directed approaches such as anti-sense oligodeoxynucleotide strategies, transcriptional regulation and gene therapy (Karp et al., Cancer Res. 54:653-665 (1994)). Therefore, apoptosis should be amenable to therapeutic intervention. In this regard, one may either stimulate or inhibit the process depending upon whether one wants to increase or decrease the rate of programmed cell death.

Transgenic Animals

Techniques that allow foreign DNA sequences to be introduced into the mammalian germ line have been developed in mice. See Manipulating the Mouse Embryo (Hogan et al.,eds., 2d ed., Cold Spring Harbor Press, 1994) (ISBN 0-87969-384-3). At present, one route of introducing foreign DNA into a germ line entails the direct microinjection of a few hundred linear DNA molecules into a pronucleus of a fertilized one-cell egg. Microinjected eggs may then be subsequently transferred into the oviducts of pseudo-pregnant foster mothers and allowed to develop. It has been reported by Brinster et al. (1985), that about 25% of the mice that develop inherit one or more copies of the micro-injected DNA.

In addition to transgenic mice, other transgenic animals have been made. For example, transgenic domestic livestock, such as pigs, sheep, and cattle. Once integrated into the germ line, the foreign DNA may be expressed in the tissue of choice at high levels to produce a functional protein. The resulting animal exhibits the desired phenotypic property resulting from the production of the functional protein.

With so many members in the ICE/CED-3 family, it is important to determine the ICE/CED-3 family member's functions-individually. Transgenic mice are an ideal model for accomplishing this by generating mutations in the genes of interest, or knocking out a particular gene. Using such models, it has already been shown that mice deficient in ICE develop normally but are resistant to endotoxic shock induced by lipopolysaccharide (LPS). This can be attributed to their defect in production of mature IL-1β (Li et al., Cell 80:401411 (1995); Kuida et al., Science 267:2000-2003 (1995)). Furthermore, ICE deficient thymocytes from knockout mice undergo apoptosis normally when stimulated with dexamethasone and γ-irradiation but are partially resistant to Fas induced apoptosis (Kuida et al., Science 267:2000-2003 (1995)), suggesting that ICE plays an important role in Fas but not dexamethasone and γ-irradiation induced apoptosis in thymocytes.

Generation of mutant mice by gene targeting technique, making crosses between mice of potential candidate genes, should provide vital information about the genetic and biochemical pathways of apoptosis. In this regard, over the last several years, transgenic animals containing specific genetic defects, e.g., those resulting in the development of, or predisposition to, various disease states, have been made. These transgenic animals can be useful in characterizing the effect of such a defect on the organism as a whole, and developing pharmacological treatments for these defects. Obtaining transgenic animals in which specific genes and proteins of the apoptotic pathway are altered or eliminated (e.g. knock-out mice) results in a better understanding of the regulation of programmed cell death.

A transgenic mouse has been made whose phenotype is similar to ALS (Gurney et al., Science 264:17721775 (1994)). The trans-gene has a mutation in superoxide dismutase (SOD). These animals have age-dependent progressive motor weakness similar to ALS in humans. A different transgenic mouse has now been made that expresses a mutant of ICE, which is a dominant negative inhibitor of the ICE pathway. (Friedlander et al., J. Exp. Med. 185:933-940 (1997)).

In light of the various biological roles of apoptosis in disease, there exists a need in the art to develop treatments addressed to modulating cell death in various pathological states, such as ALS. There also exists a need to develop transgenic animals, e.g., transgenic mice, wherein one can test the role of apoptosis in ALS. Finally, there also exists a need in the art to develop methods to test compounds directed to modifying the apoptotic condition using such transgenic animals.

### SUMMARY OF THE INVENTION

It has now been found that a dominant negative mutant of the cell death gene ICE (Caspase-1) significantly slows the symptomatic progression of ALS and delays mortality in a transgenic mouse model of ALS. This suggests the involvement of the ICE-like proteases in ALS progression and the therapeutic value of ICE inhibitors in the treatment of ALS in humans.

It has also been found that when a transgenic mouse expressing an ALS phenotype (SOD mutation) is crossed with a transgenic mouse having a mutant ICE gene, the resulting offspring have an attenuated form of ALS. This provides a new transgenic model for studying ALS and for testing new treatments for the disease.

Therefore, this invention satisfies a need in the art for finding a treatment for ALS and providing new animal models to study this disease.

The invention is first directed to a method for treating ALS. Preferably, the invention is directed to treating cell death during ALS and more preferably to treating neuronal cell death during ALS.

In an embodiment of this invention the treatment of ALS involves gene therapy to ameliorate the effects of the ICE gene. Preferably the gene therapy involves use of a mutant ICE gene comprising a DNA sequence encoding an amino acid sequence wherein the cysteine residue in the active site of ICE is replaced with a glycine. In a specific embodiment the replacement at the cysteine residue in the active site of the murine ICE is at amino acid 284 (C284G). More preferably, the gene sequence is found in plasmid pJ655 having ATCC accession number 209077 deposited in the American Type Culture Collection (10801 University Boulevard, Manassas, Va. 20110-2209, USA. on May 28, 1997 under the Budapest Treaty) or is a degenerate variant of said mutant gene.

In another embodiment of the invention, treatment of ALS involves the use of protease inhibitors selected from the group consisting of N-benzyloxycarbonyl-Val-Ala-Asp-fluoromethylketone (z-VAD.FMK), acetyl-Tyr-Val-Ala-Asp-chloromethylketone (Ac-YVAD.CMK) (SEQ ID No. 26), N-benzyloxycarbonyl-Asp-Glu-Val-Asp-flouromethylketone (z-DEVD.FMK) (SEQ ID No. 27) and Ac-YVAD-CHO (SEQ ID No. 28).

Another embodiment of the invention involves the treatment of traumatic brain injury by inhibition of the ICE cell death family. This may be done by use of ICE inhibitors or directly affecting the relevant gene as in the knockout mice of the claimed invention. A further related embodiment of the invention is drawn to reducing the formation of reactive oxygen species (ROS) by inhibiting the ICE cell death family.

The invention is further directed to a non-human transgenic animal expressing the ALS phenotype that also contains a mutant ICE gene. Preferably the non-human animal is a mouse. Such mice exhibit an increased period of time in which the disease exists and therefore live longer than an ALS mouse not expressing the dominant negative mutant ICE gene.

In a preferable embodiment of this invention, the mutant ICE gene in the transgenic animal comprises DNA encoding an amino acid sequence wherein the cysteine residue in the active site of ICE is replaced with a glycine, e.g. in the mouse (C284G). More specifically, the mutant gene is found in plasmid pJ655 having ATCC accession number 209077 or is a degenerate variant of said mutant gene.

In additional embodiments, this invention provides a method of testing compounds affecting ALS by providing a non-human animal with ALS that also has a mutant ICE gene, wherein the animal exhibits an increased resistance to ALS. One administers a compound to be tested to the transgenic animal, and determines the effect of the compound on the mortality of the animal relative to an animal with the SOD mutation but without the mutant ICE gene.

### BRIEF DESCRIPTION OF THE FIGURES

FIGS. 1A-1B. The amino acid sequence of ced-3 and ICE genes. (SEQ ID NOS: 1-2, 4 and 7-11) FIGS. 1A-1B contains a comparison of the amino acid sequences of ced-3 from C. elegans, C. briggsae and C. vulgaris with hICE, mICE and mouse nedd-2. Amino acids are numbered at the right of each row. The lines indicate gaps resulting from obtaining optimal alignment of the sequences. Residues that are conserved among more than half of the proteins are boxed. Missense ced-3 mutations are indicated above the comparison blocks showing the residue in the mutant ced-3 and the allele name. Asterisks indicate potential aspartate self-cleavage sites in ced-3. Circles indicate known aspartate self-cleavage sites in HICE. FIGS. 1A-1B also includes the sequences of mutant ICE proteins wherein the C is replaced with a G (SEQ. ID. Nos. 1-2). As indicated on the figure, the mutation in the mouse gene encodes a protein having glycine rather than cysteine at position 284 (C284G) (SEQ ID NO: 1). A similar mutation may be created in the human ICE, except the change is made at position 285 (C285G). (SEQ ID NO: 2)

FIGS. 2A-2D—DNA and amino acid sequence of wild-type and mutant murine ICE. FIG. 2A is the amino acid sequence of wild-type murine ICE (SEQ ID NO: 4). FIG. 2B is the DNA sequence of wild-type murine ICE (SEQ ID NO:5). FIG. 2C is the amino acid sequence of the mutant murine ICE (SEQ ID NO:6). FIG. 2D is the DNA sequence of the mutant murine ICE (SEQ. ID. No. 3).

FIGS. 3A-3C: Protection from permanent middle cerebral artery (MCA) occlusion-mediated infarct. Infarct protection in NSE-M17Z (black) was compared with wild-type (white) mice. FIG. 3A. Neurological grading 30 minutes and 24 hours following occlusion. Neurological grading: 0=no neurological deficits; 1=failure to extend the right forepaw; 2=circling to the contralateral side; 3=loss of walking or righting reflex. FIG. 3B. Infarct area assessed at 24 hours. FIG. 2C. Regional cerebral blood flow (rCBF), and mean blood pressure (MBP) of wild type and transgenic mice during 30 minutes of ischemia (**p<0.01).

FIG. 4: Whole brain lysates of NSE-M17Z mice are deficient in processing pro-IL-1β following systemic LPS (lipopolysaccharide) administration. LPS was injected intraperitoneally (10 μg/gr body weight) and 2 hours before sacrifice (wild type n=4, NSE-M17Z n=5). PBS was injected as a control (wild type n=3, NSE-M17Z n=6). Brains were dissected, and mature IL-1β concentration was determined using an ELISA kit specific for mature IL-1β. Results are expressed as means±SEM.

FIG. 5: DNA damage in the lesioned hemisphere of wild-type mice. Lane 1 shows the DNA size marker with 200 bp steps (M), lane 2 and 3 (T1 and T2) the DNA ladder prepared from right coronal sections 6 mm from frontal pole 24 hours after weight drop trauma, and lane 4 and 5 (S1 and S2) the DNA from the corresponding section of the right hemisphere of sham-operated animals. T1, T2, S 1 and S2 were taken from different animals.

FIG. 6: Trauma-induced elevation of mature IL-1βb levels in brain. Empty columns: sham-operated animals, black columns: traumatized animals. Data are presented as mean±SEM (n=5 or 6 in duplicate); *P<0.02 vs. contralateral hemisphere in traumatized animals; ⁺P<0.008 vs. ipsilateral hemisphere in sham-operated animals. Mature IL-1β quantification was performed as previously described using an ELISA kit (Genzyme, Cambridge, Mass.) (Hara, H., et al., Proc. Natl. Acad. Sci. USA 94:2007-2012 (1997)). Brains were removed 5 hours following trauma or sham operation, and each hemisphere without 2 mm from frontal and occipital poles were homogenized. Sham-operated mice were craniectomized but not traumatized.

FIGS. 7A-7C Total lesion volume after traumatic brain injury in NSE-M17Z transgenic mice or corresponding wild-type littermates (FIG. 7A) or in C57BL/6 mice (FIGS. 7B and 7C). Lesion size was assessed 24 hours following weight drop impact to the right hemisphere. zVAD-fmk (480 ng) was injected i.c.v. either 1 hour before trauma (13) or 1 hour after trauma (C). Total lesion volume is calculated from lesion areas determined in each of 5 coronal sections (2 mm) from anterior (2 mm behind frontal pole) to posterior (10 mm). Total lesion volume was decreased in NSE-M17Z transgenic mice and after injection of zVAD-fink 1 hour before trauma. Although total lesion volume in animals injected with zVAD-fink 1 hour after trauma was not significantly different, the size of lesion in the treated mice in the two most anterior sections (2-6 mm) was significantly reduced (P<0.05; n=5; data not shown) indicating a trend of a protective effect. Data are presented as mean±SEM (n-5-6). *P<0.05 vs. vehicle or, in case of transgenic mice, vs. wild-type littermates.

FIG. 8. Trauma-induced increase in free radical production in M17Z and wild-type mouse brain homogenates. Results are shown as 3,4-DHBA (3,4-dihydroxybenzoic acid)/4-HBA (4-hydroxybenzoic acid) ratio and represent the change in free radical production when compared to ipsilateral hemispheres of sham-operated mice. Data are presented as mean±SEM (n=7); *P=0.03.

### DETAILED DESCRIPTION OF THE PREFERRED EMBODIMENT

In the description that follows, a variety of technical terms are used.

Unless the context indicates otherwise, these terms shall have their ordinary well-recognized meaning in the art. In order to provide a clearer and more consistent understanding of the specification and claims, the following definitions are provided.

Italicized words such as “ICE” refer to the gene while non-italicized words such as “ICE” refer to the RNA or protein product encoded by the corresponding gene.

ALS or ALS-like Symptoms. As used herein, the terms “ALS or ALS-like symptoms” refers to asymmetric weakness in two or more limbs, progressing to complete paralysis. This may also be described as an age-dependent progressive motor weakness. Onset of the disease may be described by a significantly slower gait than corresponding control subjects.

Active Site As used herein, “active site” refers to the catalytic site of the ICE protein. In human ICE, the active site comprises at least amino acids 283-287, while in the murine ICE this comprises at least amino acids 282-286. The active site contains the consensus amino acid sequence QACRG (SEQ ID No. 12).

Apoptosis. As used herein, “apoptosis” refers to the process by which organisms eliminate unwanted cells. The process is regulated by a cellular program. Apoptosis may eliminate cells during normal development, aging, tissue homeostasis or following imposition of an external stress such as hypoxia or trophic factor deprivation or during a disease state such as in ALS.

Central Nervous System Damage. As used herein, “central nervous system damage refers to any injury to the central nervous system that results in programmed cell death or apoptosis of neurons. Specific examples of such damage is that which results from ALS, traumatic brain injury (TBI), Alzheimer's disease, stroke and spinal cord injury. Such examples, however, are not meant to be limiting and also include other central nervous system damage recognized by those of skill in the art to result from neuronal apoptosis.

Dominant Negative Inhibitor. As used herein, “dominant negative inhibitor” refers to a mutated version of the wild type protein, that when expressed in cells can inhibit the activity of the endogenous protein.

Expression vector. As used herein, an “expression vector” is a vector comprising a structural gene operably linked to an expression control sequence so that the structural gene can be expressed when the expression vector is transformed into an appropriate host cell. Two DNA sequences are said to be “operably linked” if the nature of the linkage between the two DNA sequences does not (1) result in the introduction of a frame-shift mutation, (2) interfere with the ability of the promoter region sequence to direct the transcription of the desired sequence, or (3) interfere with the ability of the desired sequence to be transcribed by the promoter region sequence. Thus, a promoter region would be operably linked to a desired DNA sequence if the promoter were capable of effecting transcription of that DNA sequence.

Gene therapy. As used herein, “gene therapy” means, inter alia, the ability to ameliorate or eliminate a genetic defect by altering the gene of interest or the product expressed by the gene of interest, by altering the genotype of the cell or organism of interest. For example, this may be accomplished by replacing the gene with a mutated gene, knocking out the gene of interest or inserting a different gene that produces a product that inhibits or stimulates the gene of interest or using other methods known to those of skill in the art. The manipulation of the genetic material may be accomplished either in vivo or ex vivo. These examples are not to be construed as limiting the different ways in which the gene therapy may be effected.

ICE pathway. As used herein, “ICE pathway” refers to the pathway by which interleukin-IP converting enzyme is activated and converts the pro-ILβ to mature IL-β eventually resulting in programmed cell death.

Modulating programmed cell death. As used herein, “modulating programmed cell death” should be understood to mean that one either increases or decreases cell death depending upon the desired end result.

Mutatedgene. As used herein, “mutated gene” refers to a gene containing an insertion, substitution, or deletion resulting in the loss of substantially all of the biological activity associated with the gene. For example, a mutated ICE gene may either not express the protein of interest or if the substitution is minimal may express the protein of interest, but the protein may have a loss of biological activity. The term “biological activity” is readily understood by those of skill in the art. For example, the biological activity of an enzyme relates to the ability of the enzyme to act on its appropriate substrate and effect catalysis of the reaction converting the substrate to the appropriate product. Alternatively, biological activity of a growth factor could be that activity which stimulates a target cell to divide or express a specific protein.

For the purposes of exemplification, a specific example of a gene encoding a mutation in a murine ICE protein (C284G) is presented in the specification. However, similar mutations (e.g. in the active site of ICE) in genes encoding corresponding ICE proteins from other species such as in human ICE are within the scope of this invention.

Resistant to or attenuated. As used herein “resistant to” or attenuated” means that an animal exposed to a certain treatment shows a greater degree of survivability, will live longer than the corresponding control (i.e. the treatment results in decreased lethality from the disease or condition than what is observed in the corresponding control) or will show an improvement in the disease symptoms. This does not necessarily mean that all animals will survive the treatment or that the animals will recover from the disease.

Targeting vector. As used herein “a targeting vector” is a vector comprising sequences that can be inserted into a gene to be disrupted, e.g., by homologous recombination. Therefore, a targeting vector may contain sequences homologous to the gene to be disrupted.

Transgenic. As used herein, a “transgenic organism” is an organism containing a defined change to its germ line, wherein the change is not ordinarily found in wild-type organisms. This change can be passed on to the organism's progeny and therefore the progeny are also transgenic animals. The change to the organism's germ line can be an insertion, a substitution, or a deletion in the gene of interest. The term “transgenic” also encompasses organisms containing modifications to their existing genes and organisms modified to contain exogenous genes introduced into their germ line. Thus, the term also “transgenic” also encompasses organisms where a gene has been eliminated, modified or disrupted so as to result in the elimination of a phenotypic characteristic associated with the disrupted gene (e.g. “knock-out animals”). This invention relates to non-human transgenic animals comprising a mutant ICE gene and a mutant SOD gene.

Vector. As used herein, a “vector” is a plasmid, phage, or other DNA sequence, which provides an appropriate nucleic acid environment for a transfer of a gene of interest into a host cell. Cloning vectors will ordinarily replicate autonomously in eukaryotic hosts. The cloning vector may be further characterized in terms of endonuclease restriction sites where the vector may be cut in a determinable fashion. The vector may also comprise a marker suitable for use in identifying cells transformed with the cloning vector. For example, markers can be antibiotic resistance genes.

Gene Therapy

A patient (human or non-human) suffering from ALS symptoms may be treated by gene therapy. By undertaking this approach, there should be an attenuation of the ALS symptoms. Gene therapy approaches have proven effective or to have promise in the treatment of certain forms of human hemophilia (Bontempo, F. A., et al., Blood 69:1721-1724 (1987); Palmer, T. D., et al., Blood 73:438445 (1989); Axelrod, J. H., et al., Proc. Natl. Acad. Sci. USA 87:5173-5177 (1990); Armentano, D., et a., Proc. Natl. Acad. Sci. USA 87:6141-6145 (1990)), as well as in the treatment of certain other mammalian diseases such as cystic fibrosis (Drumm, M. L., et a., Cell 62:1227-1233 (1990); Gregory, R. J., et al, Nature 347:358-363 (1990); Rich, D. P., et al., Nature 347:358-363 (1990)), Gaucher disease (Sorge, J., et al., Proc. Natl. Acad. Sci. USA 84:906-909 (1987); Fink, J. K., et al., Proc. Natl. Acad. Sci. USA 87:2334-2338 (1990)), muscular dystrophy (Partridge, T. A., et al., Nature 337:176-179 (1989); Law, P. K., et al., Lancet 336:114-115 (1990); Morgan, J. E., et al., J. Cell Biol. 111:2437-2449 (1990)), and metastatic melanoma (Rosenberg, S. A., et al., Science 233:1318-1321 (1986); Rosenberg, S. A., et al, N. Eng. J. Med. 319:1676-1680 (1988); Rosenberg, S. A., et al., N. Eng. J. Med. 323:570-578 (1990)).

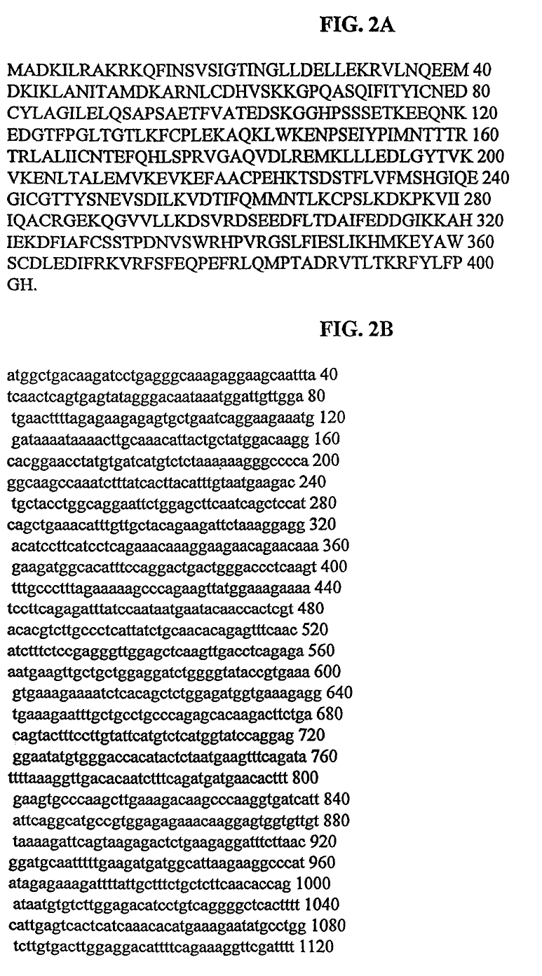

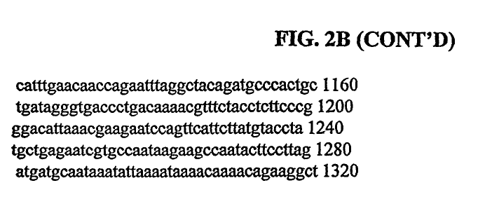

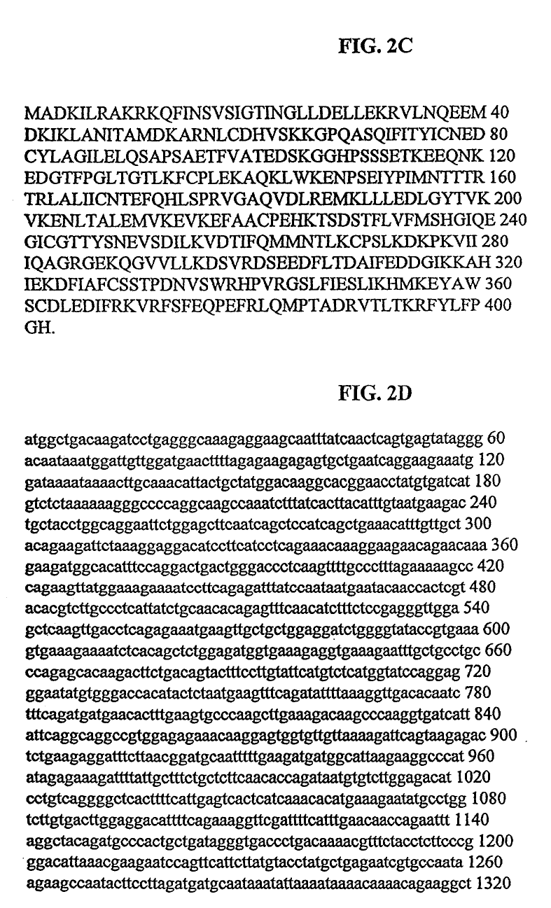

In a preferred approach, a polynucleotide having the nucleotide sequence depicted in FIG. 2D (SEQ ID NO:3), that of the cDNA clone deposited as pJ655, ATCC Accession No. 209077 or a degenerate variant of the sequence, a nucleic acid molecule encoding an ICE inhibitor, or a nucleic acid molecule complementary to said inhibitor, or an anti-sense sequence for the ICE gene may be incorporated into a vector suitable for introducing the nucleic acid molecule into cells of the mammal to be treated, to form a transfection vector.

Knowing the amino acid sequence of an ICE inhibitor, one of skill in the art may readily determine the corresponding nucleic acid sequence based on the the triplet codons for each amino acid. Conversely, knowing the DNA sequence one may readily determine the derived amino acid sequence. Furthermore, knowing the triplet codon for an amino acid, one can also readily determine degenerate variants of that triplet codon such that they still encode the same amino acid sequence.

Suitable vectors for this purpose include retroviruses and adenoviruses. Alternatively, the nucleic acid molecules of the invention may be complexed into a molecular conjugate with a virus (e.g., an adenovirus) or with viral components (e.g., viral capsid proteins).

Techniques for the formation of such vectors comprising the inhibitor nucleic acid molecule or an anti-sense sequence to the ICE gene are well-known in the art, and are generally described in “Working Toward Human Gene Therapy,” Chapter 28 in Recombinant DNA, 2nd Ed., Watson, J. D. et al., eds., New York: Scientific American Books, pp. 567-581 (1992). In addition, general methods for construction of gene therapy vectors and the introduction thereof into affected animals for therapeutic purposes may be found in the above-referenced publications, the disclosures of which are specifically incorporated herein by reference in their entirety.

In one such general method, vectors comprising the isolated mutant ICE gene are directly introduced into the cells or tissues of the affected individual, preferably by injection, inhalation, ingestion or introduction into a mucous membrane via solution; such an approach is generally referred to as “in vivo” gene therapy. Alternatively, cells or tissues, e.g., hematopoietic cells from bone marrow, may be removed from the affected animal and placed into culture according to methods that are well-known to one of ordinary skill in the art; the vectors comprising the polynucleotides may then be introduced into these cells or tissues by any of the methods described generally above for introducing isolated polynucleotides into a cell or tissue, and, after a sufficient amount of time to allow incorporation of the polynucleotides, the cells or tissues may then be re-inserted into the affected animal or a second animal in need of treatment. Since the introduction of the DNA of interest is performed outside of the body of the affected animal, this approach is generally referred to as “ex vivo” gene therapy.

For both in vivo and ex vivo gene therapy, the polynucleotides of the invention may alternatively be operatively linked to a regulatory DNA sequence, which may be a heterologous regulatory DNA sequence, to form a genetic construct as described above. This genetic construct may then be inserted into a vector, which is then directly introduced into the affected animal in an in vivo gene therapy approach, or into the cells or tissues of the affected animal in an ex vivo approach. In another preferred embodiment, the genetic construct may be introduced into the cells or tissues of the animal, either in vivo or ex vivo, in a molecular conjugate with a virus (e.g., an adenovirus) or viral components (e.g., viral capsid proteins).

The above approaches result in (a) homologous recombination between the nucleic acid molecule and the defective gene in the cells of the affected animal; (b) random insertion of the gene into the host cell genome; or (c) incorporation of the gene into the nucleus of the cells where it may exist as an extrachromosomal genetic element. General descriptions of such methods and approaches to gene therapy may be found, for example, in U.S. Pat. No. 5,578,461; WO 94/12650; and WO 93/09222.

Alternatively, transfected host cells, which may be homologous or heterologous, may be encapsulated within a semi-permeable barrier device and implanted into the affected animal, allowing passage of for example, the ICE inhibitor into the tissues and circulation of the animal but preventing contact between the animal's immune system and the transfected cells (see WO 93/09222).

Vectors

A variety of vectors have been developed for gene delivery to the nervous system and to brain tumors. These vectors derive from herpes simplex virus type 1 (HSV-1), adenovirus, adeno-associated virus (AAV) and retrovirus constructs (for review see Friedmann, T., Trends Genet 10:210-214 (1994); Jolly, D., Cancer Gene Therapy 1 (1994); Mulligan, R. C., Science 260:926-932 (1993); Smith, F. et al., Rest. Neurol. Neurosci. 8:21-34 (1995)). Vectors based on HSV-1, including both recombinant virus vectors and amplicon vectors, as well as adenovirus vectors can assume an extrachromosomal state in the cell nucleus and mediate limited, long term gene expression in postmitotic cells, but not in mitotic cells. HSV-1 amplicon vectors can be grown to relatively high titers (10⁷ transducing units/ml) and have the capacity to accommodate large fragments of foreign DNA (at least 15 kb, with 10 concatemeric copies per virion). AAV vectors (rAAV), available in comparable titers to amplicon vectors, can deliver genes (<4.5 kb) to postmitotic, as well as mitotic cells in combination with adenovirus or herpes virus as helper virus. Long term transgene expression is achieved by replication and formation of “episomal” elements and/or through integration into the host cell genome at random or specific sites (for review see Samulski, R. J., Current Opinion in Genetics and Development 3:74-80 (1993); Muzyczka, N., Curr. Top. Microbiol. Immunol. 158:97-129 (1992)). HSV, adenovirus and rAAV vectors are all packaged in stable particles. Retrovirus vectors can accommodate 7-8 kb of foreign DNA and integrate into the host cell genome, but only in mitotic cells, and particles are relatively unstable with low titers. Recent studies have demonstrated that elements from different viruses can be combined to increase the delivery capacity of vectors. For example, incorporation of elements of the HIV virion, including the matrix protein and integrase, into retrovirus vectors allows transgene cassettes to enter the nucleus of non-mitotic, as well as mitotic cells and potentially to integrate into the genome of these cells (Naldini, L. et al., Science 272:263-267 (1996)); and inclusion of the vesicular somatitis virus envelope glycoprotein (VSV-G) increases stability of retrovirus particles (Emi, N. et al., J. Virol. 65:1202-1207 (1991)).

HSV-1 is a double-stranded DNA virus which is replicated and transcribed in the nucleus of the cell. HSV-1 has both a lytic and a latent cycle. HSV-1 has a wide host range, and infects many cell types in mammals and birds (including chicken, rat, mice monkey, and human) Spear et al., DNA Tumor Viruses, J. Tooze, Ed. (Cold Spring Harbor Laboratory, Cold Spring Harbor, N.Y., 1981) pp. 615-746. HSV-1 can lytically infect a wide variety of cells including neurons, fibroblasts and macrophages. In addition, HSV-I infects post-mitotic neurons in adult animals and can be maintained indefinitely in a latent state. Stevens, Current Topics in Microbiology and Immunology 70: 31(1975). Latent HSV-1 is capable of expressing genes.

AAV also has a broad host range and most human cells are thought to be injectable. The host range for integration is believed to be equally broad. AAV is a single stranded DNA parvovirus endogenous to the human population, making it a suitable gene therapy vector candidate. AAV is not associated with any disease, therefore making it safe for gene transfer applications (Cukor et al., The Parvovituses, Ed. K. I. Berns, Plenum, N.Y., (1984) pp. 33-36; Ostrove et al., Virology 113: 521 (1981)). AAV integrates into the host genome upon infection so that transgenes can be expressed indefinitely (Kotin et al., Proc. Natl. Acad. Sci. USA 87: 221 (1990); Samulski et al., Embo J. 10: 3941 (1991)). Integration of AAV into the cellular genome is independent of cell replication which is particularly important since AAV can thus transfer genes into quiescent cells (Lebkowski et al., Mol. Cell. Biol. 8:3988 (1988)).

Both HSV and AAV can deliver genes to dividing and non-dividing cells. In general, HSV virions are considered more highly infectious that AAV virions, with a ratio of virus particles: infectious units in the range of 10 for HSV (Browne, H. et al., J. Virol. 70:4311-4316(1996)) and up to thousands for AAV (Snyder, R. O. et al., In Current Protocols in Human Genetics, Eds. Dracopoli, N. et al., John Wiley and Sons: New York (1996), pp. 1-24), and both having a broad species range. Still, each virion has specific trophisms which will affect the efficiency of infection of specific cell types. The recent identification of a membrane receptor for HSV-1 which is a member of the tumor necrosis factor alpha family (Montgomery, R. I. et al., 21st Herpes Virus Workshop Abstract #167 (1996)) indicates that the distribution of this receptor will affect the relative infectability of cells, albeit most mammalian cell types appear to be injectable with HSV-1. AAV also has a very wide host and cell type range. The cellular receptor for AAV is not known, but a 150 kDA glycoprotein has been described whose presence in cultured cells correlates with their ability to bind AAV (Mizukami, H. et al., Virology 217:124-130 (1996)).

Protease Inhibitors

ICE has been identified as a cysteine protease and peptide aldehyde inhibitors of ICE have been described (Thomberry et al., Nature 356:768-774 (1992). Additionally, other peptide inhibitors of the ICE family delay motor neuron death in vitro and in vivo (Milligan et al., Neuron 15:385-393 (1995); Hara et al., Proc. Natl. Acad. Sci. USA 94:2007-2012 (1997)). These inhibitors have been shown to arrest programmed cell death of motorneurons in vivo and in vitro during the period of naturally occurring cell death accompanying development (Milligan et al., Neuron 15:385-393 (1995)). The inhibitors effects have also been shown to reduce ischemic and excitotoxic neuronal damage during reperfusion following filamentous middle cerebral occlusion (Hara et al., Proc. Natl. Acad. Sci. USA 94:2007-2012 (1997)). The peptide inhibitors used in these two different experiments included N-benzyloxycarbonyl-Val-Ala-Asp-fluoromethylketone (z-VAD.FMK), acetyl-Tyr-Val-Ala-Asp-chloromethylketone (Ac-YVAD.CMK),N-benzyloxycarbonyl-Asp-Glu-Val-Asp-flouromethylketone (z-DEVD.FMK) and Ac-YVAD-CHO. z-VAD-FMK (SEQ ID No. 29) and z-DEVD.FMK—Enzyme Systems Products—Dublin, Calif.; Ac-YVAD-CMK and YVAD-CHO—Bachem Biosciences—King of Prussia, Pa.).

Thus, the ICE protease family plays an important role in mammalian neuronal apoptosis.

The claimed invention provides a method of treating ALS symptoms and further provides a transgenic mouse model to study the disease. The transgenic mouse model of the invention comprises a mouse with attenuated ALS symptoms that provides, inter alia, a model and/or test system for investigators to manipulate and better understand the mechanisms of apoptosis and ALS. In particular, a better understanding is gained concerning the role of the ICE gene and the ICE pathway. Such a model, allows the investigator to test various drugs where physiological responses are altered in the mouse, and thereby determine more effective therapies to treat the underlying mechanism of ALS. Thus, the transgenic animals of this invention are also useful as animal models to study apoptosis and ALS

Therefore, invention also provides, a method of screening compounds, comprising: providing the compound to a transgenic non-human animal having a mutant SOD-1 gene and a mutant ICE gene and then determining the effect of the compound on apoptosis of said animal; and correlating the effect of the compound with increases or decreases in apoptosis.

The compounds to be tested can be administered to the animal having ALS and a mutant ICE gene in a variety of ways well known to one of ordinary skill in the art. For example, the compound can be administered by parenteral injection, such as subcutaneous, intramuscular, or intra-abdominal injection, infusion, ingestion, suppository administration, and skin-patch application. Moreover, the compound can be provided in a pharmaceutically acceptable carrier. See “Remington's Pharmaceutical Sciences” (1990). The effect of the compound on apoptosis and ALS can be determined using methods well known to one of ordinary skill in the art.

These aspects of the invention (i.e. those relating to the testing of compounds affecting apoptosis or ALS) are useful to screen compounds from a variety of sources. Examples of compounds that can be screened using the method of the invention include but are not limited to rationally designed and synthetic molecules, plant extracts, animal extracts, inorganic compounds, mixtures, and solutions, as well as homogeneous molecular or elemental samples. Establishing that a compound has an effect in the mutant animals has predictive value relating to that compound's effect in other animals, including humans. Such predictive values provide for initial screening of therapeutically valuable drugs.

The invention, therefore, provides a method of screening compounds, comprising: providing a transgenic non-human animal demonstrating ALS symptom having mutant SOD and ICE genes, said animal exhibiting the an attenuated form of ALS, administering a compound to be tested to the transgenic animal; determining the effect of the compound on the properties of interest in said animal; and correlating the effect of the compound on the mouse with the effect of said compound in a control animal.

Having now generally described the invention, the same will be more readily understood through reference to the following examples which are provided by way of illustration. The general description of the invention, as well as the following examples, are not intended to be limiting of the present invention.

### EXAMPLES

It has previously been demonstrated that binding of endogenously produced mature IL-1β to its type-1 receptor plays an important role in apoptosis (Friedlander et al., J. Exp. Med. 184:717-724 (1996)). It has also been shown that replacing the cysteine in the active site of ICE with a glycine in the mouse (C284G) obliterates its ability to mediate cell death (Miura et al., Cell 75:653-660 (1993)). The residue in the active site is required for the IL-1β convertase and the autoprocessing activity of ICE (Gu et al., EMBO J. 14:1923-1931 (1995)).

### Example I

### Mutant ICE Protects Dorsal Root Ganglion (DRG) Neurons

Survival of DRG neurons in culture requires the presence of trophic factors which include nerve growth factor and serum. In the absence of trophic factor support, DRG neurons undergo apoptosis (Davies et al., Development 101:185-208 (1987))). To determine whether a mutant ICE can inhibit DRG neuronal death induced by trophic factor deprivation, primary cultures of chicken embryonic DRG neurons were microinjected with a construct of the fused mutant ICE^{C284G}-lacZ gene under the control of the β-actin promoter (β-actin-M17Z).

Methods

Screening of cDNA Library

Standard techniques of molecular cloning were used as described (Sambrook et al. Molecular Cloning: A Laboratory Manual, Second Ed., Cold Spring Harbor, N.Y.: Cold Spring Harbor Laboratory Press (1989)), unless otherwise indicated. A human ICE cDNA was obtained by polymerase chain reaction (PCR) using the human ICE sequence (Thomberry et al., Nature 356, 768-774 (1992)). This cDNA was used as a probe to screen a mouse thymus cDNA library (Stratagene, LaJolla, Calif.). The filters were hybridized in 5×SSPE, 20% formamide, 0.02% Ficoll, 0.02% bovine serum albumin, 0.02% polyvinylpyrrolidone, 1% SDS at 40° C. for 2 days and washed in 1×SSPE and 0.5% SDS for 20 min, twice at room temperature and twice at 45° C.

Plasmid Construction

pJ415 was constructed by first inserting a 5'400 bp Bglll-BamHl crmA fragment into the BamHl site of the pBabe/puro vector and then inserting the remaining 1 kb BamHl crmA fragment into the 3′ BamHl site in a sense direction. pJ436 was constructed by inserting an EcoRl-Sall bcl-2 fragment into the EcoRI-Sall sites of the pBabe/puro vector (Morgenstern et al., Nucl. Acids Res. 18:3587-3596 (1990)). To construct fusion genes, the E.coli β-galactosidase gene was taken from the plasmid 407-794.Z (Picard et al, EMBO J. 6:3333-3340 (1987)) by digestion with BamHl and cloned into pBlue-script vector (BSlacZ). Various 5′ deletion fragments of mICE (pJ348) were obtained by PCR. PCR was performed by using synthetic primers (M10 and T3 primer for the whole mICE construct, M11 and T3 primer for the P20/P10 construct, M11 and M13 for the P20 construct, M12 and T3 primer for the P10 construct) and pJ348 as a template. The primer sequences were as follows: M10-AAGTCGACGCCATGGCTGACAAGATCCTGAGGG (SEQ ID No. 13), M11-AAGTCGACGCCATGAACAAAGAAGATGGCACAT (SEQ ID No. 14); M12-AAGTCGACGCCATGGGCATTAAGAAGGCCCATA (SEQ ID No. 15); M13-TTCCCGGGTCATCTTCAAAAATTGCATCCG (SEQ ID No. 16). The amplified fragments were digested with Sall and Smal and then cloned into Sall-Smal sites of BSlacZ. BSced38Z was made by first inserting a Smal-digested PCR product of ced-3 cDNA (primers used were M18 and M19; M18-AACCCGGGAGGCCTCCATGATGCGTCAAGATAGAAG (SEQ ID No. 17); M19-AACCCGGGACGGCAGAGTTTCGTGCTTCCG) (SEQ ID No. 18 into BSlacZ. BSM 10Z (mICE-IacZ in pBluescript II vector), BSM11Z (P20/P10-IacZ in pBluescript II vector), BSM19Z (P20-IacZ cloned in pBluescript II vector), BSM12Z (P10-IacZ cloned in PBluescript II vector), and BSced38Z (ced-3-IacZ cloned in pBluescript II vector) were digested with Xhol-Notl, blunt ended by Klenow fragment, and then cloned into pβactSTneoB (Miyawaki et al., 1990) (digested with Sall and blunt ended by Klenow fragment) individually, and the resulted plasmids were named pβactM10Z, pβactM11Z, pβactM12Z, and pβactced38Z, respectively. To mutate the glycine residue to a serine residue in the active domain of mICE, the PCR product of primers m8p/s (ATTCAGGCCTCCAGAGGAGAGAAAC) and mice 8 (GGCACGATTCTCAGCATAGGT), using pJ348 as a template, was digested with Sphl and Smal and then cloned into the Sphl-Smal sites of BSM10Z (pJ483). To mutate the cysteine residue to a glycine residue in the active domain of mICE, the PCR product of M10 and M15 (CAAGGCCTGCCTGAATAATGATCACCTT), using pJ348 as a template, was digested with Sall and Stul, then cloned into BSM10Z that was digested with Sphl and blunt ended by T4 DNA polymerase, and then digested with Sall (BSM17Z). To mutate the glycine residue to aserine residue in the active domain of ced-3, the PCR products of the C-terminal portion of CED-3 (primers used were M19 and M20; M20, GCAGGCCTGTCGATCGGAACGTCGTGACAATGGATI) and the N-terminal portion of CED-3 (primers used were M18 and M21; M21, ACAGGCCTGCACAAAAACGATTTT) were digested with Stul and Smal and then cloned into Smal site of BSlacZ (BSced37Z). pJ483, BSM17Z, and BSced37Z were digested with Xhol and Notl, blunt ended by Klenow fragment, and then cloned into pβactSTneoB individually, and the resulted plasmids were named pJ485, pβactM17zs, and pβactced37Z, respectively. (Miura et al., Cell 75:653-660 (1993)).

The experiments were performed essentially as described by Gagliardini et. al. (Science 263:826-828 (1994)). Primary cultures of chicken embryonic DRG neurons were isolated under sterile conditions from day 10 embryos (Spafas, Preston, Conn.). DRG's were dissociated by incubating in trypsin for 15 minutes at 37° C. and trituration. Dissociated neurons were plated on poly-L-lysine (Sigma, 30 mg/ml for 1 hour) and laminin (Sigma, 20 mg/ml for 2 hours) coated chamber slides. DRG neurons were cultured for 2 days in F12 medium (Gibco) containing 10% fetal bovine serum (Hyclone), penicillin (100 U/ml, Gibco), streptomycin (100 mg/ml, Sigma), and 5 mM cytosine b-D-arabinose (Sigma), supplemented with NGF (10 ng/ml, Sigma).

Neuron injection was performed with an Eppendorf microinjector (model 5242), with glass micropipettes loaded with 1 mg/ml plasmid DNA in TE buffer and 5% rhodamine dye (rhodamine-isothiocyanite labeled dextran, 10 kDa; Molecular probes, Eugene, Oreg.), dissolved in 0.2 M KCl. The construction of the fused mutant ICE (C284G)-lacZ plasmid (β-actin-M17Z) was described by Miura et al (Cell 75:653-660 (1993). Three hours after injection, the NGF containing medium was replaced with NGF-free and serum-free medium in the presence of sufficient mouse monoclonal antibody against NGF (Boehringer Mannheim, Indianapolis, Ind.). The medium was changed daily. Live injected neurons were counted on days 0, 3, and 6.

Neurons were co-injected with rhodamine-isothiocyanate dextran as a marker and with Hoechst dye to determine neuronal nuclear morphology. Following trophic factor removal, control neurons microinjected with the b-actin-lacZ construct survived 22.5 and 6.0% after 3 and 6 days in culture, respectively. No significant difference was detected when compared to cells injected with dye alone. In contrast, neurons injected with b-actin-M17Z survived 85.0 and 81.0% after 3 and 6 days in culture, respectively. These results showed that the mutant ICE gene inhibits DRG neuronal cell death induced by trophic factor deprivation, suggesting that mutant ICE may be able to suppress the activities of wild type ICE or ICE-like proteases.

### Example 2

### Generation of Transgenic Mice Expressing the Mutant ICE (C284G) Protein

To determine whether the mutant ICE gene can also act as an inhibitor of apoptosis in vivo, and to further evaluate its mechanism of action, transgenic mouse lines expressing the fused mutant ICE^{C284G}-lacZ gene under the control of the neuron specific enolase promoter (NSE-M17Z) were established.

pNSE-M17Z-lacZ construct was made by digesting pNSE-lacZ with SalI and ClaI which removed a 0.8 kb SalI/ClaI fragment. The SalI/ClaI digested pNSE-lacZ vector was ligated with a 2 kb salI/ClaI insert from BSM17Z which contains the mutant ICE (C284G) and the part of lacZ which was removed in the SalI/ClaI digest of the pNSE/lacZ vector. The resulting construct was named pJ655. To generate transgenic mice, pJ655 was linearized by XmnI digestion and gel purified. Fourteen transgenic mice lines were generated by DNX (Princeton, N.J.). Founder mice were SV-129/C57BL/6 hybrid. Initially 5 lines were selected (7506, 7512, 7516, 7538, and 7539) based on highest DNA copy number in the genome.

Tail DNA was isolated and genotyping was performed using the following PCR primers targeted to the Ice/lacZ fusion (M17Z-F: 5′TGCCCAAGCTTGAAAGACAAGCCC3′ (SEQ ID No. 24), lacZ-R: 5′CTGGCGAAAGGGGGATGTGCTG3′) (SEQ ID No. 25). X-gal staining was performed by removing the vertebral column and sectioning it in a sagital plane. Tissue was fixed for 5 minutes on ice (0.2% glutaraldehyde in 0.1M phosphate buffer, 2% formaldehyde, 5 mM EGTA pH 7.3, 2 mM₂) followed by three 30 minute washes at 4° C. (0.1M phosphate buffer pH 7.3, 2 mM MgCl₂, 0.1% sodium deoxycholate, 0.2% NP40). The tissue was then stained overnight with X-gal at 37° C. (rinse solution with 1 mg/ml X-Gal in DMSO, 5 mM K ferrocyanide, 5 mM K ferricyanide), and then sectioned in a cryostat (40 μm). Photomicrographs were taken in a light microscope (100×) under oil immersion.

Transgenic mice expressing either the lacZ or bcl-2 genes under control of the NSE promoter have been well characterized, and transgene expression has been detected throughout the nervous system (Martinou et al., Neuron 13:1017-1030 (1994); Forss-Petter et al., Neuron 5:187-197 (1990); Farlie et al., Proc. Natl. Acad. Sci. USA 92:4397-4401 (1994)). PCR was used for genotyping the NSE-M17Z transgenic mice, and protein expression was detected by X-gal staining. Founder mice from five different lines were crossed with C57BL/6 mice. The expression of NSE-M17Z was detected in the first and second generation offsprings which were used in some of the experiments described below.

### Example 3

### Mutant ICE Acts in vivo as a Dominant Negative Inhibitor of ICE

It was next evaluated whether the mutant ICE^{C284G} may act as a dominant negative ICE inhibitor. Ice knockout mice were almost completely defective in processing pro-IL-1β and ICE is the only protease identified so far that can process pro-IL-1β (Kuida et al, Science 267:2000-20002 (1995); Li et al., Cell 80:401-411 (1995). If the mutant Ice transgenic mice have a defect in secreting mature IL-1b, this would provide strong evidence that mutant ICE^{C284G} can act as a dominant negative inhibitor of ICE.

Systemic injection of lipopolysaccharide (LPS) induces release of mature IL-1β. ICE knockout mice generated by gene-targeting technique were unable to release mature IL-1β upon LPS stimulation (Kuida et al, Science 267:2000-20002 (1995); Li et al., Cell 80:401-411 (1995). To determine if mutant ICE^{C284G} transgenic mice are also defective in secreting mature IL-1β, LPS was injected intraperitoneally into the mutant ICE^{C284G} transgenic mice and the levels of mature IL-1β were determined in whole brain lysates using an ELISA kit that specifically detects mature IL-1β. Following the systemic LPS challenge, whole brain lysates of mutant ICE^{C284G} transgenic mice contained 74.7% less mature IL-1β as compared to that of LPS-injected wild type mice. In control wild type mice injected intraperitoneally with PBS, there was low but detectable levels of mature IL-1β in the brain (4.0 pg/g brain) whereas this cytokine was undetectable in the brain lysate of PBS (phosphate buffered saline) injected mutant ICE^{C284G} mice (FIG. 4). Thus, mutant ICE^{C284G} can act as an effective inhibitor of pro-IL-1β processing, strongly suggesting that mutant ICE^{C284G} is a dominant negative inhibitor of ICE itself.

### Example 4

### NSE-M17Z Transgenic Mice are Resistant to Cerebral Ischemic Injury

To determine if ICE plays a role in apoptosis induced by ischemic insult and if the mutant ICE^{C284G} may act to reduce ischemic brain injury, it was investigated whether the mutant ICE^{C284G} transgenic mice were protected in a mouse focal cerebral ischemia model where apoptotic cell death has been reported (Li et al, Mol. Brain Res:28L164-168 (1995). Permanent middle cerebral artery (MCA) occlusion was performed in 14 wild-type and 11 transgenic mice, progeny of 5 different founder mice (7509, 7512, 7516, 7538, and 7539). Experiments were done as described by Hara et al. (J. Cereb. Blood Flow Metab. 16:605-611 (1996), except that the occlusion of the MCA was continuous for 24 hours. Neurological grading was as follows: 0=no neurological deficits; 1=failure to extend the right forepaw; 2=circling to the contralateral side; 3=loss of walking or righting reflex. All experiments were done in a double blinded fashion. Values shown as mean±s.e.m.

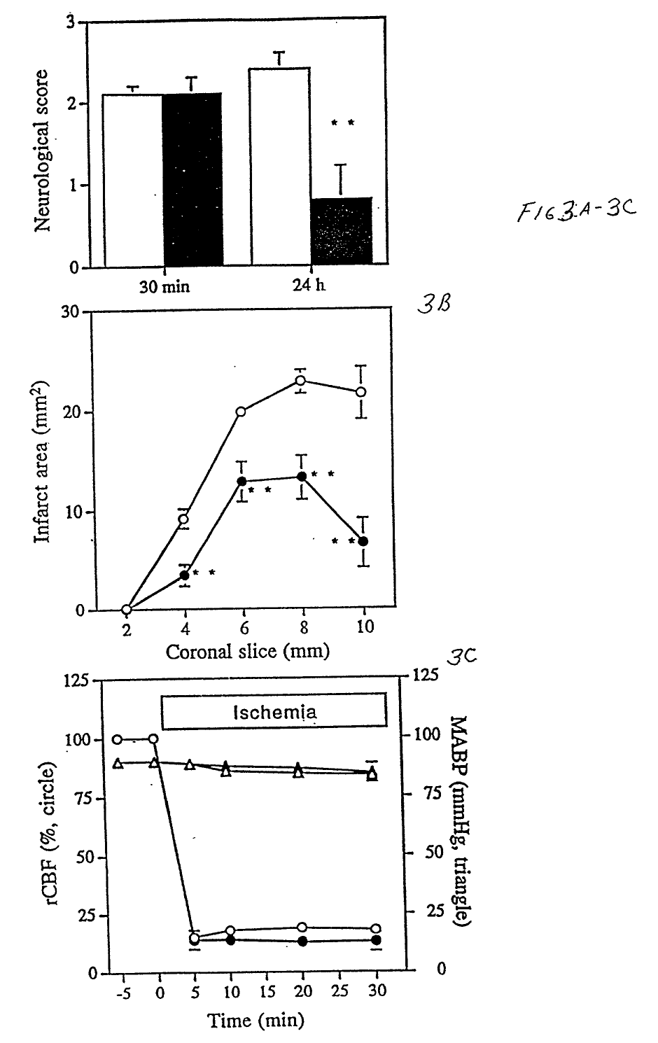

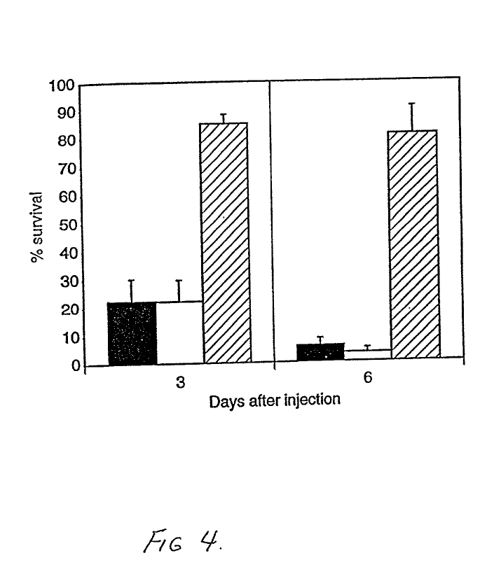

Mice were scored neurologically 30 minutes and 24 hours following the occlusion. In the initial 30 minute evaluation, there was no significant difference in the neurological score. During the ensuing 24 hours, however, wild-type mice remained impaired, whereas transgenic mice improved neurologically (FIG. 3A). Mice were sacrificed at 24 hours, and infarct volume was quantified with 4% 2,3,5-triphenyltetrazolium chloride (TTC). Infarct volume was significantly smaller in NSE-M17Z [66±11 mm³ (n=11)] when compared to the wild-type litter-mate mice [125±5 mm³ (n=14)] (FIG. 3B). Physiologic parameters were recorded in a separate set of transgenic and wild-type mice. Blood pressure, arterial blood gases (PO₂, PCO₂, and blood pH), regional cerebral blood flow, and body temperature did not significantly differ in the two sets of mice, before and throughout thirty minutes of ischemia (FIG. 3C). Thus, expression of mutant ICE^{C284G}, a dominant negative inhibitor of ICE, protects neurons from ischemic insult.

Summary of Examples 1-4

Mutant ICE^{C284G} inhibits apoptosis in two different species (chicken and mouse) and under the control of two different promoters (β-actin and NSE). Evidence has been presented that mutant ICE^{C284G} acts as a dominant negative inhibitor of ICE by inhibiting processing of pro-IL-1β. X-ray crystallography analysis showed that ICE exists as a dimer of two p20 and two p10 subunits processed from two p45 precursor molecules (Wilson et al., Nature 370:270-275 (1994)). Expression of catalytically inactive mutant of ICE may result in formation of inactive dimers which will inhibit endogenous wild type ICE function.

Since DRG neurons from Ice knockout mice were protected from trophic factor deprivation-induced apoptosis as were DRG neurons from NSE-M17Z transgenic mice, it is believed that, at least in DRG neurons, the mutant ICE^{C284G} prevented neuronal cell death by inhibiting ICE activity. This is consistent with previous experiments demonstrating that the addition of the IL-1receptor antagonist, a naturally existing IL-1β antagonist, inhibited mouse DRG neuronal cell death induced by trophic factor deprivation, suggesting that release of mature IL-1β processed by ICE plays an active role in apoptosis induced by trophic factor deprivation (Friedlander et al., J. Exp. Med. 184:717-724 (1996)). The role of ICE-like proteases in apoptosis induced by cerebral ischemia in a mouse permanent focal stroke model was determined. NSE-M17Z transgenic mice suffered less tissue injury and less behavioral changes as a result of ischemic injury as compared to the wild type mice. Although it cannot be ruled out that the reduction of neuronal damage by mutant ICE was due to its ability to inhibit other members of the Ice family, evidence suggests that ICE itself plays an important role in cerebral ischemia-induced cell death. Elevated levels of IL-1β are detected following cerebral ischemia (Lui et al., Stroke 24:1746-1751 (1993)). In addition, intraventricular administration of the IL-1β receptor antagonist decreases infarct size following permanent middle cerebral artery (MCA) occlusion (Relton et al., Brain Res. Bull. 29:243-246 (1992)). It has also demonstrated that endogenously produced mature IL-1b plays an important role in hypoxia-mediated apoptosis in vitro (Friedlander et al., J. Exp. Med. 184:717-724 (1996)). These results suggest the involvement of ICE and of mature IL-1b receptor binding in the mechanism of ischemia-induced cell death. The results further corroborate the notion that ICE plays an important role in apoptosis induced by ischemic injury. Following exposure to certain death stimuli, the ICE cell death cascade is activated. As demonstrated apoptosis may be inhibited by blocking the ICE cell death cascade, either the activation of pro-ICE, the function of active ICE, or the product of ICE activity which is mature IL-1β (Gagliardini et al., Science 263:826-828 (1994); Friedlander et al., J. Exp. Med. 184:717-724 (1996) Boudreau et al., Science 267:891-893 (1995)). It cannot be ruled out, however, that mutant ICE^{C284G} may also cross-inhibit other cell death gene products, since subunits of different ICE family members sharing significant sequence homology may bind to each other forming hetero-oligomeres (Gu et al., EMBO J 14:1923-1931 (1995)). Since embryonic development of the mutant ICE^{C284G} and of Ice knockout mice are normal and no significant defect in embryonic apoptosis was uncovered (Kuida et al., Science 267:2000-2002 (1995); Li et al., Cell 80:401411 (1995)), a nonredundant function of ICE in developmental apoptosis has been ruled out. It has been demonstrated here that transgenic mice expressing a dominant negative mutant ICE are significantly protected from neuronal cell death induced by ischemic insult, suggesting that ICE may play an important role in pathological cell death. Our results suggest that ischemic-induced injury, and possibly other disorders featuring apoptosis, can be treated with inhibitors aimed at modulating the activity of the ICE protease family in order to reduce tissue injury and preserve brain function.

### Example 5

### Inhibition of ICE Activity in vivo

To determine if inhibition of ICE activity in vivo might halt the progression of the ALS-like syndrome in the mutant SOD-1 mice, 5 female NSE-M17Z mice from one transgenic founder mouse (Friedlander et al., J. Exp. Med. 185:933-940 (1997)) having a mutant ICE gene (as described above) were crossed with one mutant SOD(G93R) male mouse (Gurney et al., Science 264:1772-1775 (1994)). The genotypes of the progeny from these crosses were determined for the carriers of the mutant ICE and mutant SOD transgenes by PCR. Litter mates of SOD(G93R) and SOD(G93R); mutant ICE were monitored for the times of disease onset and death.

The onset of the disease was scored as the appearance of significantly slower gait than that of Litter mates and/or limb paralysis. The end point was scored as death or when flipped on its side and is unable to get up in 30 sec. The scorers were completely unaware of the genotypes of the mice or their birthdates.

It was found that although the timing of the disease onset in the mutant SOD and mutant SOD/mutant ICE (M17Z) transgenics is not different, the double transgenic mice survive significantly longer (27 days) than the mutant SOD mice alone (11.7 days) following the onset of the disease (Table 1). These results indicated that expression of the dominant negative inhibitor of ICE in neurons of

TABLE 1
Delay of mortality in SOD/G93R transgenic mice by expression of
a dominant negative inhibitor of ICE

| | | Length of | |
|---|---|---|---|
| | Onset (days) | Disease | Mortality |
| SOD mean (n = 24) | 237.7 ± 4.4 | 11.7 ± 1.6 | 249.3 ± 3.8 |
| SOD/M17Z mean (n = 22) | 243.0 ± 5.6 | 27.0 ± 3.5 | 270.0 ± 7.0 |
| Unpaired t test (two tailed) | p = 0.464 | *p = 0.0002 | *p = 0.0115 |

*indicate significant difference

mutant SOD mice is able to slow significantly the time of the symptomatic progression of this disease and delays mortality. These results suggest the involvement of the ICE-like proteases in the disease progression in this ALS mouse model and possible therapeutic value of ICE inhibitors in the treatment of ALS in humans.

Furthermore, based on these observations one may treat ALS patients using a recombinantly made ICE mutant protein. The mutant protein is obtained by using an appropriate expression vector followed by isolation of the protein, all of which uses methods readily known to those of skill in the art expressing. The treatment comprises contacting the cells of a patient (human or non-human) in need of treatment for ALS or ALS-like symptoms with the recombinantly made mutant ICE protein. Such contact may be made either in vivo or in vitro.

### Example 6

### Screening of Compounds that Affect ALS Using Transgenic Mice with a Mutant ICE Gene and SOD Gene

The transgenic mice comprising a mutant mice ICE gene and mutant SOD gene exhibit a delayed mortality and increased timecourse for ALS. This may be related to the inhibition of the ICE gene product, ICE-related proteases or the ICE cell death pathway. Using the transgenic mouse of the invention to screen compounds allows the pre-clinical determination of combinations of compounds which would be beneficial in treating ALS in affected individuals.

For example, with the effects of the ICE gene product blocked in the transgenic animal of the invention, a drug may further attenuate the ALS symptoms. In an animal without the mutant ICE gene, the effect of the drug of interest may not be determinable because any amelioration of symptoms it might produce are overcome by the effects of the ICE gene product. This problem should be minimized in a transgenic SOD mutant mouse that also expresses the mutant ICE gene.

Alternatively, if a drug if a drug potentiates the symptoms of ALS, its use may be contra-indicated for therapy. Thus, the mutant mice may be used for screening compounds for treating ALS and its related symptoms

Compounds to be screened for activity can be administered t6 the transgenic mice with the mutant ICE/SOD genes using pharmaceutically acceptable methods. See Remington's Pharmaceutical Sciences (1990). For example, the compound to be screened can be administered at various concentrations by parenteral injection, infusion, ingestion, and other suitable methods in admixture with a pharmaceutically acceptable carrier. The effect of various concentrations of the screened compound on increasing or decreasing the symptoms and mortality to ALS is measured. This is determined relative to control SOD mutant transgenic animals without the mutant ICE gene, transgenic animals that have not been administered the compound and wild-type animals without either the SOD or the ICE mutant genes.

A significant delay in mortality or increase in the time in which the ALS symptoms are expressed in mice with the mutant ICE/SOD genes, following treatment with a screened compound may be indicative that the compound would exhibit beneficial effects in the treatment of ALS. Such a benefit may be obtained either alone or in combination with a compound that inhibits ICE activity.

Particularly preferred compounds for screening are those compounds known to inhibit activities of ICE in vitro or any other candidate for treating ALS.

### Example 7

### Treatment of ALS—Gene Therapy

A patient (human or non-human) with ALS symptoms is treated by gene therapy such that the effects of the ICE gene product are blocked. This may be accomplished by using the mutant ICE gene as described in Example 2. Alternatively, a human mutant ICE gene is used that contains a mutation in the active site of the ICE, e.g. the cysteine may be replaced with a glycine at amino acid 285, resulting in a C285G mutant rather than a C284G mutant (as in the mouse). The sequence of the human ICE gene can be obtained in the art (Thornberry, N. A., Nature 356:768-774 (1992). Other mutations in the active site or elsewhere in the gene may also be appropriate. An appropriate vector such as adenovirus or herpes virus is chosen to infect the patient and the mutant gene is thereby directly introduced into the cells of the affected individual.

Alternatively, cells or tissues may be removed from the affected individual and placed into culture. The mutant ICE gene is then introduced into the cultured cells or tissues and then re-inserted into the patient.

### Example 8

### Treatment of ALS—Protease Inhibitors

A patient (human or non-human) with ALS symptoms is treated with protease inhibitors such that the effects of the ICE gene product are blocked and the ALS symptoms are attenuated. The protease inhibitors are selected from the group consisting of N-benzyloxycarbonyl-Val-Ala-Asp-fluoromethylketone (z-VAD.FMK), acetyl-Tyr-Val-Ala-Asp-chloromethylketone (YVAD.CMK), N-benzyloxycarbonyl-Asp-Glu-Val-Asp-flouromethylketone (z-DEVD.FMK) and Ac-YVAD-CHO.

### Example 9

### Post-Traumatic Brain Injury and Oligonucleosomal DNA Degradation

Necrotic and apoptotic cell death both play a role mediating tissue injury following brain trauma. The Interleukin-1β converting enzyme (ICE) is activated and oligonucleosomal DNA fragmentation is detected in traumatized brain tissue. Reduction of tissue injury and free radical production following brain trauma was achieved in a transgenic mouse expressing a dominant negative inhibitor of ICE in the brain. Neuroprotection was also conferred by intracerebroventricular administration of the caspase inhibitor N-benzyloxycarbonyl-Val-Ala-Asp-fluoromethylketone (zVAD-fmk). These results indicate that inhibition of ICE-like caspases reduces trauma-mediated brain tissue injury. In addition, in vivo functional interaction between ICE-like caspases and free radical production pathways, implicating free radical production as a downstream mediator of the caspase cell death cascade has been demonstrated.

A transgenic mouse expressing a dominant negative inhibitor of ICE, which has the active site cysteine substituted for glycine (C285G), in neurons under the control of the neuron specific enolase promotor (NSE-M17Z) was generated (Fiedlander, R. M., et al., J. Exp. Med. 185:933-940 (1997)). ICE was activated following cerebral ischemia, and expression of the M17Z transgene decreased ischemia-induced cerebral infarct, as well as mature IL-1β production (Hara, H., et al., Proc. Natl. Acad. Sci. USA 94:2007-2012 (1997); Hara, H., et al., J. Cereb. Blood Flow Metab. 17:370-375 (1997); Friedlander, R. M., et al., J. Exp. Med. 185:933-940 (1997)). Furthermore, synthetic peptide inhibitors of ICE-like caspases reduced infarct size following focal cerebral ischemia (Hara, H., et al., Proc. Natl. Acad. Sci. USA 94:2007-2012 (1997); Loddick, S. A., et al., NeuroReport 7:1465-1468 (1996)). ICE-mediated cell death also plays a role in the progression of amyotrophic lateral sclerosis (ALS) in the familial ALS transgenic mouse model (Friedlander, R. M., et al., Nature 388:31 (1997)). The above evidence implicates ICE-like caspases as important mediators of cell death in a variety of neurological conditions.

Although apoptosis has been detected in experimental brain injury models (Colicos, M. A. & Dash, P. K., Brain Res. 739:120-131 (1996); Yakovlev, A. G. et al., J. Neurosci. 17:7415-7424 (1997); Sinson, G., et al., J. Neurosurg. 86:511-518 (1997)), this is the first study to specifically investigate the role of the ICE cell death pathway in vivo following cerebral trauma. Inhibition of this apoptotic pathway might attenuate traumatic cerebral injury. It is shown that ICE is activated in a traumatic brain injury model and that inhibition of the ICE cell death cascade, both by genetic and pharmacologic means reduces traumatic brain injury.

Methods

Traumatic Brain Injury.

Brain trauma experiments and lesion quantification were performed essentially as described (Chan, P. H., et al., Ann. NY Acad. Sci. 738:93-103 (1994)). Spontaneously ventilating adult mice were initially anesthetized with halothane in 70% N₂O and 30% O₂, and fixed in a stereotactic frame. Before trauma, an atraumatic craniectomy was performed by removing the right parietal bone posterior to the coronal, lateral to the sagittal, and anterior to the lambdoid suture. Laterally, the craniectomy was extended to the insertion of the temporalis muscle. A piston which was 3 mm in diameter, and had an excursion of 3 mm was then placed over the craniectomy defect. A 20 g weight was dropped inside a cylinder from a height of 150 mm onto the piston (final speed v=1.70 m/s). Twenty-four hours after trauma, brains were removed and sectioned into five coronal (2 mm) slices and stained with 2% 2,3,5-triphenyl tetrazolium chloride (TTC) as described for a weight drop trauma model (Chan, P. H., et al., Ann. NY Acad. Sci. 738:93-103 (1994)). Surgical procedure as well as quantification of lesion size was performed by an investigator naive to the animal identity. Lesion volume was calculated using an image analysis system (M4, Imaging Research, St. Catherines, Ontario, Canada) from the total lesion volume integrated from the volumes of each single section, after subtracting the volume of the deficient cortex as caused by piston penetration. The volume of the piston penetration was calculated as that of a cylinder (pr²h=3.14×1.5²×3 mm³=21.2 mm³), where r is the radius of the piston, and h the depth of penetration. The trauma protocol was approved by the IACUC. The NSE-M17Z and wild-type littermate mice were bred from C57BL/6 background, and the wild-type mice used for zVAD-fink injection experiments were C57BL/6 (Taconic Farms, Germantown, N.Y.). NSE-M17Z mice were genotyped as previously described (Friedlander, R. M., et al., J. Exp. Med. 185:933-940 (1997)). zVAD-fmk (480 ng) or vehicle (DMSO 0.4%) was injected i.c.v. (2 μl; bregma −0.9 mm lateral, −0.1 mm posterior, −3.1 mm deep) 1 hour before or 1 hour after trauma.

DNA Fragmentation Analysis.

DNA was end-labeled with [³²P]ddATP, electrophoresed on a 2% agarose gel and autoradiographed. For analysis of DNA damage, tissue samples corresponding to the striatum in slice 3 were obtained 24 hours after trauma. DNA was isolated using a kit (Puregene), digested with DNAse-free RNAse (Boehringer Mannheim) and extracted with phenol-chloroform. DNA was reprecipitated in ethanol, pelleted and resuspended. DNA concentration was quantified by absorbance at 260 nm. For visualization of damaged DNA, strand breaks were end-labeled with [³²P]ddATP (Tilly, J. L. & Hsueh, A. J. W., J. Cell Physiol. 154:519-526 (1993); MacManus, J. P., et al., Mol. Neurosci. 5:493-496 (1995)). Three μg of DNA were used for labeling, electrophoresed on a 2.0% agarose gel (agarose 3:1, Amresco), and detected by autoradiography.

Statistics.

Data are presented as mean±SEM. Statistical comparisons were made by Student's t-test. For comparison of lesion size two-way ANOVA followed by Tukey posthoc tests was applied. P<0.05 was considered statistically significant. For statistical evaluation by Student's t-test, data on OH production was log transformed.

Results

To determine whether apoptotic cell death develops as a consequence of direct impact brain injury, traumatized tissue was examined for the presence of oligonucleosomal DNA fragmentation. Oligonucleosomal DNA degradation was detected following experimental traumatic brain injury.

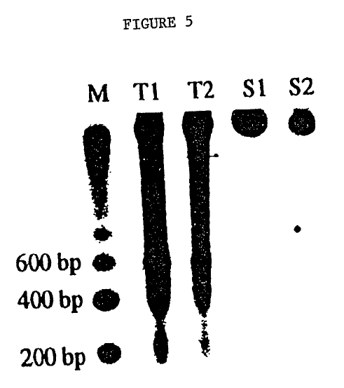

In the lesioned hemisphere, extensive DNA fragmentation was found 24 hours following trauma. DNA damage was not detected in brain tissue from sham-operated mice (FIG. 5). DNA fragments appeared on agarose gels as a ladder reflecting oligonucleosomal DNA fragmentation superimposed upon a smear reflecting random DNA degradation. Random DNA degradation results from necrotic cell death, whereas oligonucleosomal DNA fragmentation occurs following apoptotic cell death. This result indicated that both necrotic as well as apoptotic cell death pathways are activated and likely play a role in experimental TBI.

### Example 10

### ICE is Activated Following Traumatic Brain Injury

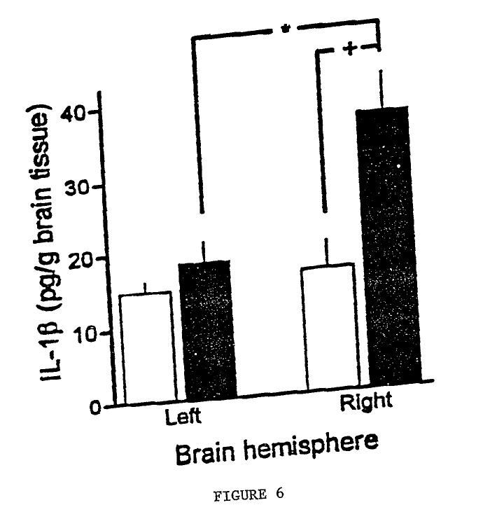

Pro-IL-1β processing requires functional ICE activity as demonstrated in ICE knock-out mice following lipopolysaccharide challenge (Li, P., et al., Cell 80:401-411 (1995); Kuida, K., et al., Science 267:2000-2003 (1995)). Therefore, detection of mature IL-1β has been employed as direct evidence for ICE activation (Miura, M., et al., Proc. Natl. Acad. Sci. USA 92:8318-8322 (1995); Hara, H., et al., Proc. Natl. Acad. Sci. USA 94:2007-2012 (1997); Hara, H., et al., J. Cereb. Blood Flow Metab. 17:370-375 (1997); Troy, C. M., et al., Proc. Natl. Acad. Sci. USA 93:5635-5640 (1996); Friedlander, R. M., et al., J. Exp. Med. 185:933-940 (1997); Li, P., et al., Cell 80:401-411 (1995); Kuida, K., et al., Science 267:2000-2003 (1995)). In traumatized mice, brain tissue mature IL-1β levels were significantly increased to 37.3±5.2 pg/g brain tissue as compared with 16.6±3.6 pg/g brain tissue in the ipsilateral hemisphere of sham operated mice. Mature IL-1β levels in the contralateral hemisphere (18.4±2.8 pg/g tissue) did not significantly differ from corresponding levels in sham-operated mice (14.9±1.4 pg/g tissue) (FIG. 6). Elevated levels of mature IL-1β were not detected 24 hours following injury. Early detection of ICE activity is consistent with that previously reported during apoptosis (Enari, M., et al., Nature 380:723-726 (1996); Hara, H., et al., Proc. Natl. Acad. Sci. USA 94:2007-2012 (1997); Hara, H., et al., J. Cereb. Blood Flow Metab. 17:370-375 (1997)).

### Example 11

### ICE Family Inhibition Reduces Traumatic Tissue Injury.

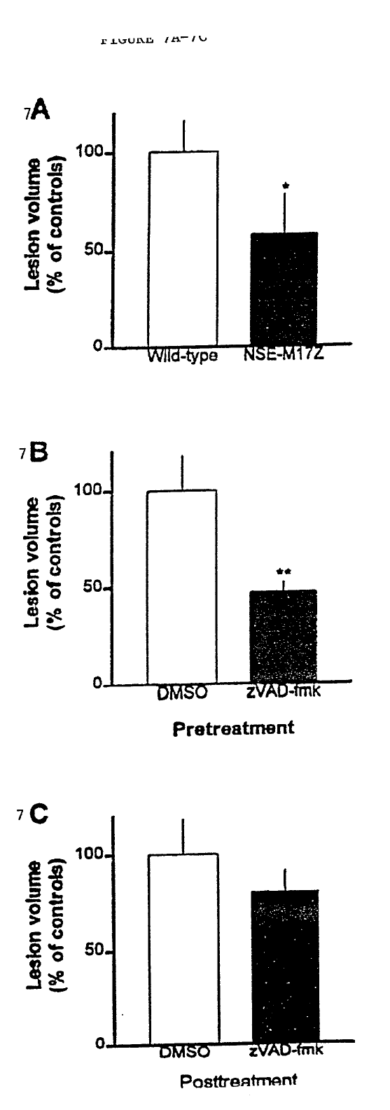

Since ICE is activated following TBI, it was evaluated whether ICE inhibition might reduce trauma-mediated brain injury. Previously it has been demonstrated that the M17Z transgene behaves as a dominant negative inhibitor of ICE (Hara, H., et al., J. Cereb. Blood Flow Metab. 17:370-375 (1997); Friedlander, R. M., et al, J. Exp. Med. 185:933-940(1997)). To evaluate whether ICE inhibition might attenuate brain trauma mediated damage, lesion size in NSE-M17Z transgenic mice with that of wild-type littermates 24 hours post-impact was compared. Total lesion volume in the NSE-M17Z mice, 24 hours following trauma, was significantly reduced by 42.3% when compared to wild-type mice (FIG. 7A). The M17Z mutant ICE gene confers tissue protection following traumatic injury, implicating ICE-like caspases as mediators of traumatic-induced cell death. Protection from cerebral ischemia mediated injury in the NSE-M17Z transgenic mouse correlates with protection by synthetic peptide ICE family protease inhibitors (Hara, H., et al., Proc. Natl. Acad. Sci. USA 94:2007-2012 (1997); Friedlander, R. M., et al., J. Exp. Med. 185:933-940 (1997)). It was evaluated whether zVAD-fink (a general ICE family protease inhibitor) would diminish traumatic tissue damage as was demonstrated in the NSE-M17Z transgenic mouse. Wild-type mice were injected with zVAD-fink (480 ng) into the lateral cerebral ventricle 1 hour prior to impact. Total lesion volume 24 hours following trauma in the treated mice was significantly reduced by 53% when compared to the vehicle-injected mice (FIG. 7B). Moreover, lesion volume was reduced by 19% if zVAD-fink was administered one hour following trauma (FIG. 7C). Statistical significance was only reached in the anterior two out of five slices, suggesting that a therapeutic window exists for the treatment of TBI with caspase inhibitors. These results further confirm that the caspase family plays a role in traumatic brain injury-mediated apoptosis and suggest that strategies of ICE family inhibition may be useful to treat the consequences of brain trauma.

### Example 12

### Free Radical Production is a Downstream Mediator of the ICE Cell Death Cascade

Reactive oxygen species (ROS) are potent mediators of cell death. Therefore, whether ICE-like caspases play a role in the modulation of free radical production was investigated.

Little is known regarding the actual mechanisms by which ICE activation mediates cell death. ICE can activate caspase-3, and caspase-3 has been recently shown to activate a DNase which mediates apoptotic cell death (Tewari, M., et al., Cell 81:801-809 (1995); Enari, M., et al., Nature 391:43-50 (1998)). Clearly additional pathways must be recruited following ICE activation playing a role mediating cell death. Free radical production has been implicated as an important downstream mediator of cell death (Greenlund, L. J. S., et al., Neuron 14:303-315 (1995); Schulz, J. B., et al., J. Neurosci. 16:4696-4706 (1996)). Free radical production increases following traumatic brain injury (Globus, M. Y., et al., J. Neurochem. 65:1704-1711 (1995)). For this reason it was evaluated whether ICE inhibition in vivo might attenuate free radical production.

Methods

Hydroxyl radical detection. Hydroxyl radical production was determined in mice that underwent weight drop trauma, as well as in sham-operated mice. Fifteen minutes prior to craniectomy, mice were intraperitoneally injected with 400 mg 4-hydroxybenzoic acid (4-HBA)/kg body weight, and sacrificed thirty minutes after trauma/craniectomy. Brains were removed, and the hemispheres were separated minus 2 mm of the frontal and occipital lobes. Tissue was homogenized in 0.2 M perchloric acid (1:5, w:v) at 4° C., vortexed and centrifuged (12,000 rpm, 15 min, 4° C.). Supernatant was analyzed using HPLC/EC. The HPLC system consisted of a dual piston pump (ESA model 480 pump; ESA Inc., Chelmsford, Mass.), two pulse dampers in series, a refrigerated autosampler (CMA/200, CMA/Microdialysis) and a Coulochem II (model 5200A, ESA Inc.) electrochemical detector. Data collection was performed using an ESA501 data station. Analytes were separated on a SuperODS 5 cm×4.6 mm, 2 mm column (TosoHaas; Montgomeryville, Pa.) kept at 29° C. The mobile phase delivered at 1 ml/min consisted of 100 mM NaH₂PO₄, pH 2.8 with phosphoric acid, 6.5% methanol (v/v). Analytes were detected using dual coulometric electrode analytical cell (model 5011, ESA Inc.). The potentials applied to the first and the second electrodes were +150 mV and +700 mV, respectively. 3,4-Dihydroxybenzoic acid (3,4-DHBA) was detected on the first electrode, and 4-HBA on the second. Potential of the guard cell (model 5020, ESA Inc.), placed between the pump and the injection valve was set at +200 mV. Under these conditions, the limit of detection for 3,4-DHBA was about 1 pg on the column, and the chromatogram was completed in less than 6 min.

Results

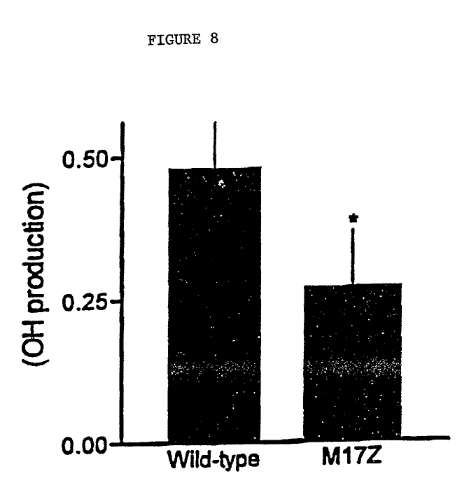

Elevation of free radical production following trauma was significantly decreased by 43% in NSE-M17Z transgenic mice compared to its wild-type littermates. No difference of baseline free radical production was detected between sham-operated wild-type and NSE-M17Z mice (FIG. 8). The results implicate ROS production as a downstream mediator of the ICE cell death cascade.

Summary and Discussion of Examples 9-12.

These results provide evidence that ICE-like caspase-family-induced apoptosis plays an important role mediating post-traumatic cerebral damage. First, it is demonstrated that ICE activation follows traumatic injury. Second, reduced injury in a transgenic mouse expressing a dominant negative ICE inhibitor following trauma is documented. Third, it is shown that injection of a non-selective ICE-like caspase family inhibitor (zVAD-fmk) 1 hour before or 1 hour after brain trauma reduced the volume of traumatic lesion in this brain injury model. The results confirm reports indicating that apoptosis, as well as caspases, contribute to cellular injury following experimental TBI (Colicos, M. A. & Dash, P. K., Brain Res. 739:120-131 (1996); Yakovlev, A. G. et al., J. Neurosci. 17:7415-7424 (1997)). Further, this data extends previous findings by implicating ICE-like caspase activation in TBI, and by demonstrating tissue protection from TBI by inhibiting the ICE cascade.

Caspases are involved in the induction and execution of programmed cell death in acute and chronic neurological disorders (Holtzman, D. M. & Deshmukh, M., Nature Medicine 3:954-955 (1997)). ICE itself does not appear to play a significant role in developmental apoptotic cell death as demonstrated in ICE knock-out mice (Li, P., et al., Cell 80:401-411 (1995); Kuida, K., et al., Science 267:2000-2003 (1995)), and in the NSE-M17Z transgenic mouse Friedlander, R. M., et al., J. Exp. Med. 185:933-940 (1997)). However, in view of the prominent role that ICE and ICE-like caspases play in ischemia, ALS, and trauma, it is proposed that alternate apoptotic pathways might be preferentially activated during pathological and developmental apoptosis (Hara, H., et al., Proc. Natl. Acad. Sci. USA 94:2007-2012 (1997); Hara, H., et al., J. Cereb. Blood Flow Metab. 17:370-375 (1997); Friedlander, R. M., et al., J. Exp. Med. 185:933-940 (1997); Friedlander, R. M., et al., Nature 388:31 (1997)).

There is substantial in vitro evidence implicating formation of ROS in certain forms of neuronal apoptosis, including those induced by staurosporine and growth factor withdrawal (Greenlund, L. J. S., et al., Neuron 14:303-315 (1995); Prehn, J. H. M., et al., J. Neurochem. 68:1679-1685 (1997)). Expression of p53 induces apoptosis in hippocampal pyramidal neurons, and p53 activates genes involved in the generation of oxidative stress (Jordan, J., et al., J. Neurosci. 17:1397-1405 (1997); Polyak, K., et al., Nature 389:300-305 (1997)). In some forms of apoptosis, generation of ROS are an early signal in the apoptotic cascade (Greenlund, L. J. S., et al., Neuron 14:303-315 (1995)). Recently it was shown in a BDNF deprivation model that increased peroxynitrite formation causes protein nitration, DNA fragmentation, and apoptotic cell death (Estévez, A. G., et al., J. Neurosci. 18:923-931 (1998)). In other forms of apoptosis ROS generation is a downstream event, since ICE inhibitors block their generation (Schulz, J. B., et al., J. Neurosci. 16:4696-4706 (1996)). The results presented above are the first to demonstrate that ICE is activated following traumatic brain injury, and that in vivo inhibition of the ICE cell death family reduces formation of ROS. ICE-mediated free radical formation therefore appears to be a downstream effector of caspase-induced apoptosis in vivo. Traumatic-induced injury, as well as cell death in other disorders featuring apoptosis, may be treated with inhibitors aimed at modulating ICE family activity to reduce brain injury and preserve brain function.

All art mentioned herein is incorporated by reference into the disclosure. Having now fully described the invention by way of illustration and example for purposes of clarity and understanding, it will be apparent to those of ordinary skill in the art that certain changes and modification may be made in the disclosed embodiments and such modifications are intended to be within the scope of the present invention. As examples, the preferred embodiments constitute only one form of carrying out the claimed invention.

What is claimed is:

1. A method of treating ALS or ALS-like symptoms comprising inhibiting ICE by gene therapy.

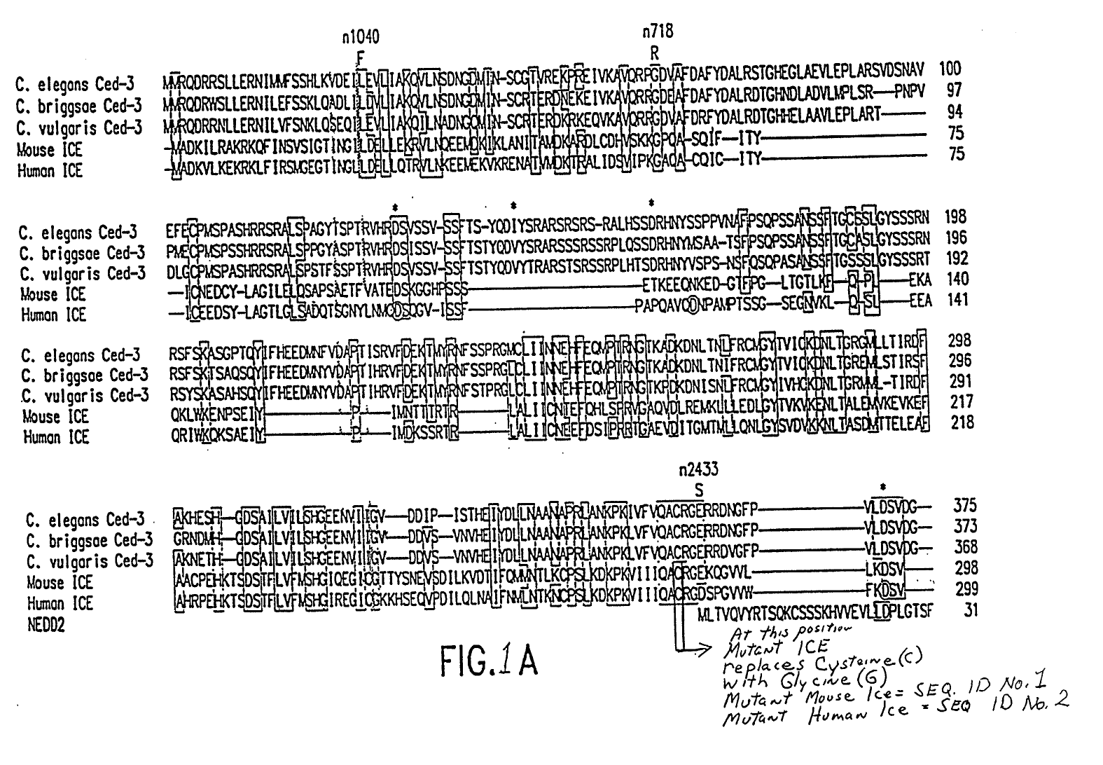

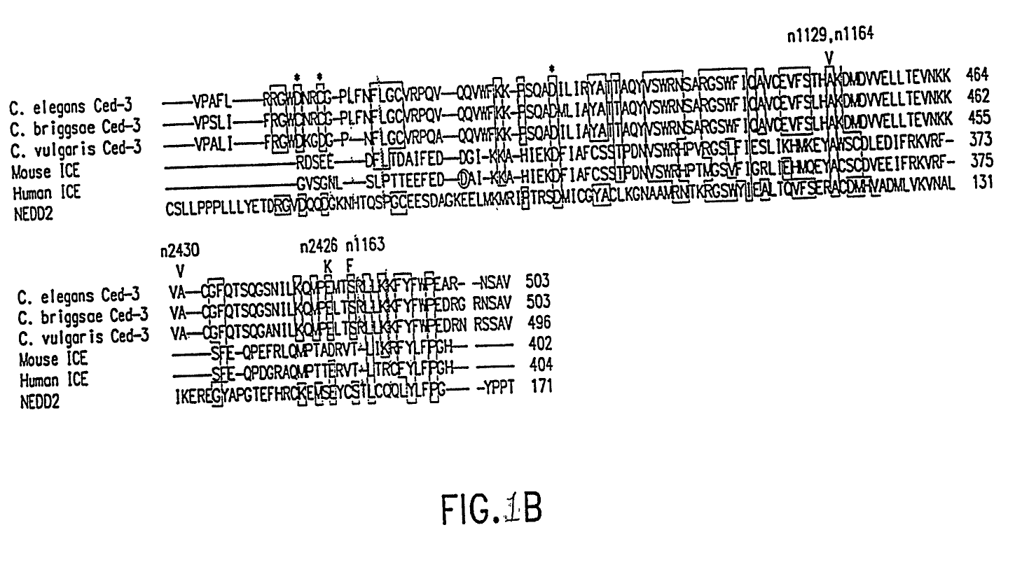

2. The method of claim 1, wherein said gene therapy uses a mutant ICE gene, said gene encoding the amino acid sequence of SEQ. ID. No. 1 or SEQ. ID. No. 2 in FIGS. 1A-1B.

3. The method of claim 1, wherein said gene therapy uses a mutant gene encoding an amino acid change in the active site of ICE.

4. The method of claim 3, wherein said mutant gene is a mouse gene that encodes a C284G mutant ICE.

5. The method of claim 3, wherein said mutant gene is found in pJ655 (ATCC accession no. 209077).

6. The method of claim 3, wherein said mutant gene is a human gene that encodes a C285G mutant ICE.

7. The method of claim 2, wherein said gene is a degenerate variant of the mutant ICE gene encoding the amino acid sequence of SEQ ID. Nos. 1 or SEQ. ID. No. 2 in FIG. 1.

8. The method of claim 1, wherein said gene therapy uses a mutant ICE gene, said gene having the DNA sequence of FIG. 2D (SEQ ID. No. 3).

9. The method of claim 8, wherein said gene comprises a degenerate variant of the DNA sequence of FIG. 2D (SEQ ID. No. 3).

10. A method for modulating programmed cell death accompanying ALS comprising contacting a cell with modulating amounts of the mutant ICE gene product.

11. A transgenic non-human animal comprising a mutant ICE gene and a mutant SOD gene.

12. The transgenic non-human animal as claimed in claim 9, wherein said animal is a mouse.

13. The transgenic non-human animal as claimed in claim 8, wherein said transgenic animal has attenuated ALS symptoms.

14. A transgenic non-human animal model for the study of ALS wherein said model comprises an animal with a mutant ICE gene and a mutant SOD gene.

15. The progeny of said transgenic non-human animal claimed in claim 9.

16. A method of screening compounds for treating ALS, comprising:
(a) providing a transgenic non-human animal having a mutant ICE gene and a mutant SOD gene, said transgenic animal exhibiting attenuated symptoms of ALS;
(b) administering a compound to be tested to said transgenic animal;
(c) determining the effect of said compound on ALS; and
(d) correlating the effect of said compound on ALS in said animal with the effect of said compound on animals with said mutant SOD gene but without said mutant ICE gene.

17. A method of obtaining a new transgenic non-human animal having a mutant ICE gene and a mutant SOD gene, said transgenic animal characterized by attenuated ALS symptoms, said method comprising:
a) mating a first transgenic animal having a mutant ICE gene to a second transgenic animal having a mutant SOD gene;
b) obtaining DNA from the progeny of said mating; and
c) verifying that the genotype of said progeny contains the mutant ICE and SOD genes.

18. The progeny of the transgenic animals obtained by the method of claim 13.

19. A method for delaying mortality from ALS comprising treating subjects having ALS or ALS symptoms with inhibitors of ICE selected from the group consisting of N-benzyloxycarbonyl-Val-Ala-Asp-fluoromethylketone (z-VAD.FMK), acetyl-Tyr-Val-Ala-Asp-chloromethylketone (Ac-YVAD.CMK), N-benzyloxycarbonyl-Asp-Glu-Val-Asp-flouromethylketone (z-DEVD.FMK) and Ac-YVAD-CHO.

20. A method for attenuating or preventing apoptosis resulting from traumatic brain injury comprising treating a patient in need of such treatment by inhibiting an ICE-like caspase.

21. A method for reducing the formation of reactive oxygen species following brain trauma comprising treating a patient in need of such by inhibiting an ICE-like caspase.
